(19) United States
(12) Patent Application Publication
(10) Pub. No.: US 2024/0398754 A1
(43) Pub. Date: Dec. 5, 2024

(54) VERATRALDEHYDE, PIPERONYL ALCOHOL, ETHYL 2-AMINO BENZOATE, ISOAMYL CINNAMATE, OR DIETHYL MALONATE AS FOOD ADDITIVES WITH ANTI-INFLAMMATORY PROPERTIES

(71) Applicant: NATIONAL UNIVERSITY OF IRELAND, MAYNOOTH, County Kildare (IE)

(72) Inventors: John C. STEPHENS, County Cavan (IE); Ronan BERGIN, County Kildare (IE); Andrew E. HOGAN, County Dublin (IE)

(21) Appl. No.: 18/698,130

(22) PCT Filed: Oct. 24, 2022
(86) PCT No.: PCT/EP2022/079672

§ 371 Date: Apr. 3, 2024

(30) Foreign Application Priority Data
Oct. 22, 2021 (GB) 2115216.0

(51) Int. Cl. A61K 31/36; A61K 9/00; A61K 31/11; A61K 31/216; A61K 31/225; A61K 31/245

(52) U.S. Cl. CPC A61K 31/36; A61K 9/0053; A61K 31/11; A61K 31/216; A61K 31/225; A61K 31/245

(57) ABSTRACT

The invention relates to agents for use in preventing or treating an inflammatory disease or condition in a subject, wherein the anti-inflammatory agent is veratraldehyde, or piperonyl alcohol, or ethyl 2-amino benzoate, or isoamyl cinnamate, or diethyl malonate; and uses of methyl isoeugenol for arthritis.

### FIELD OF THE INVENTION

The invention relates to food additives with anti-inflammatory properties. In particular, the invention relates to food additives aimed at treating or preventing type 2 diabetes and insulin resistance. The invention also relates to methods and compositions comprising said additives.

### BACKGROUND TO THE INVENTION

Worldwide, high blood glucose kills about 3.4 million people annually, and the WHO projects that deaths due to diabetes will double between 2005 and 2030. A major driver of Type 2 diabetes is the body's ineffective use of insulin and major risk factors are diet, obesity and physical inactivity. Furthermore, although historically, Type 2 Diabetes was only seen in adults, in recent years, Type 2 diabetes has become more frequent in children.

There are a number of known measures that can be taken to prevent or delay the onset of Type 2 diabetes including: eating a healthy diet, getting plenty of exercise, maintaining a healthy body weight and not smoking. However, despite these measures, the number of people diagnosed with Type 2 diabetes, or the precursor condition insulin resistance is increasing. Given how common widespread insulin resistance and type 2 diabetes are becoming, and the burden that will place on healthcare system there is a need for simple, accessible means of reducing the risk of disease.

Therefore, an aim of the invention is to provide further treatments or preventative measures for insulin resistance, Type 2 diabetes, or other diseases having similar aggravating factors.

### STATEMENTS OF INVENTION

According to a first aspect of the present invention, there is provided an anti-inflammatory agent for use in treating or preventing an inflammatory disease or condition in a subject, optionally wherein the anti-inflammatory agent is veratraldehyde, or piperonyl alcohol, or methyl isoeugenol, or citronellol, or isoamyl cinnamate, or ethyl 2-amino benzoate, or diethyl malonate, or ethyl salicylate, benzaldehyde, or citronellal, or trans-cinnamaldehyde, or farnesol, or limonene, or linalool, or geraniol.

In one embodiment, the anti-inflammatory agent is provided for oral consumption. In a preferred embodiment, the anti-inflammatory agent is a food additive.

Insulin resistance and type 2 diabetes are known to be intrinsically linked with chronic inflammation. Tumour Necrosis Factor (TNF) and interleukin-1 (IL-1) are cytokines normally expressed during infection, however, during insulin resistance and type 2 diabetes, both TNF and IL-1 are elevated. As elevated levels of TNF and IL-1 have been associated with driving insulin resistance and Type 2 Diabetes, reducing levels of TNF and IL-1 can have a preventative effect. Developing anti-inflammatory food additives can represent an efficient way to prevent or treat inflammatory disorders, such as reducing the risk of insulin resistance and Type 2 Diabetes. Additionally, food additives represent an easy and efficient way to prevent certain health problems and can be used to ensure complete nutrition, for example fortified plant-based products or adding fluoride to drinking water. Using the agents in the form of food additives can also make supplements accessible to consumers where the food additives of the present invention can be readily included into the diet of people at risk of, or suffering from, an inflammatory related disorder, such as insulin resistance or Type 2 diabetes.

The anti-inflammatory agent can advantageously provide therapeutic or preventative effects for several other inflammatory-related diseases or conditions, which may include prediabetes, gestational diabetes, metabolic disorders, cardiovascular disease (CVD), connective tissue diseases, arthritis and other joint diseases, periodontitis, gout, spondyloarthritis, uveitis, hidradenitis suppurativa, systemic lupus erythematosus (SLE), chronic obstructive pulmonary disease (COPD), psoriasis, rheumatoid arthritis, psoriatic arthritis, inflammatory bowel disease, liver disease, pain, neurological and psychiatric disorders, neurodegenerative disorders (e.g. Alzheimer's, dementia), depression, stress, narcolepsy, COVID 19, viral infections.

In an alternative aspect of the invention, there is provided a method of treating or preventing an inflammatory disorder in a subject comprising oral consumption of food comprising an anti-inflammatory agent as a food additive.

In a further aspect of the invention, there is a provided use of an anti-inflammatory agent described herein in the manufacture of a medicament for use in treating or preventing an inflammatory disease or condition in a subject.

The anti-inflammatory agent, which may be a food additive suitable for human consumption, may comprise one of the following compounds:

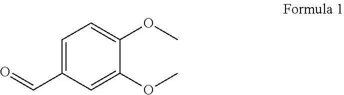

Formula 1 is veratraldehyde (methylvanillin, vanillin methyl ether, or 3, 4-dimethoxybenzaldehyde) and has the CAS number: 102-14-9;

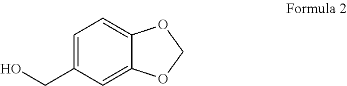 [SMILES: OCc1ccc2c(c1)OCO2]

Formula 2 is piperonyl alcohol (benzo[d][1,3]dioxol-5-ylmethanol) and has the CAS number: 495-76-1;

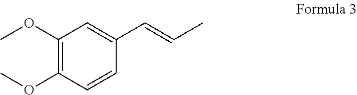

Formula 3 is methyl isoeugenol (1,2-dimethoxy-4-prop-1-enylbenzene) and has the CAS number 93-16-3;

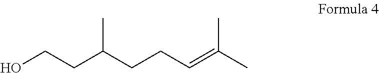

Formula 4 is citronellol (3,7-Dimethyloct-6-en-1-ol) and has the CAS number 106-22-9;

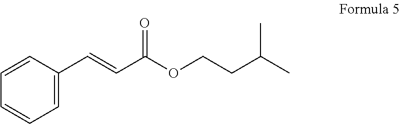 [SMILES: CC(C)CCOC(=O)/C=C/c1ccccc1]

Formula 5 is isoamyl cinnamate (3-methylbutyl (E)-3-phenylprop-2-enoate) and has the CAS number 7779-65-9;

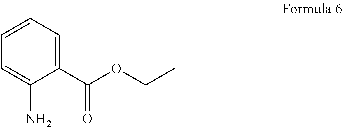

Formula 6 is ethyl 2-amino benzoate and has the CAS number 87-25-2;

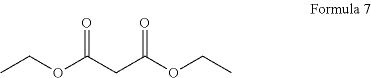

Formula 7 is diethyl malonate and has the CAS number 105-53-3;

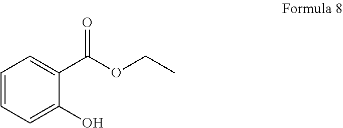

Formula 8 is ethyl salicylate (ethyl 2-hydroxybenzoate) and has the CAS number 118-61-6;

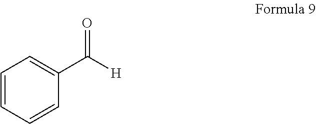

Formula 9 is benzaldehyde and has the CAS number 100-52-7;

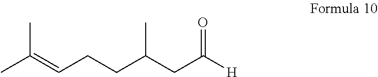

Formula 10 is citronellal (3,7-Dimethyl-6-octenal) and has the CAS number 106-23-0;

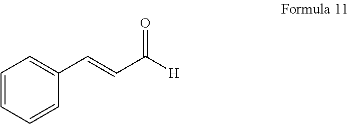

Formula 11 is trans-cinnamaldehyde and has the CAS number 104-55-2;

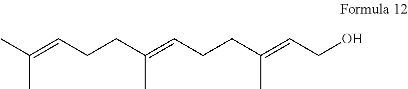 [SMILES: CC(C)=CCC/C(C)=C/CC/C(C)=C/CO]

Formula 12 is farnesol and has the CAS number 4602-84-0;

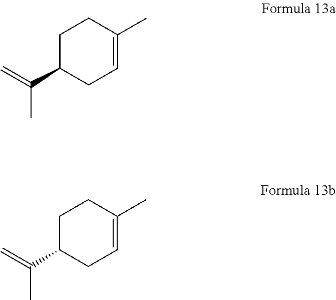

Formula 13a or 13b is limonene having has the CAS number (S): 5989-54-8 (R): 5989-27-5, wherein 13a is ((S)-1-methyl-4-(prop-1-en-2-yl)cyclohex-1-ene and 13b is (R)-1-methyl-4-(prop-1-en-2-yl)cyclohex-1-ene));

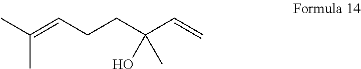

Formula 14 is linalool and has the CAS number 78-70-6;

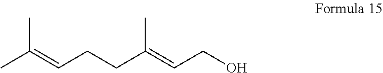

Formula 15 is geraniol ((E)-3,7-dimethylocta-2,6-dien-1-ol), and has the CAS number 106-24-1.

One anti-inflammatory agent may be used in the present invention. In one embodiment, one anti-inflammatory agent is used and is selected from veratraldehyde, piperonyl alcohol, methyl isoeugenol, citronellol, isoamyl cinnamate, ethyl 2-amino benzoate, diethyl malonate, ethyl salicylate, benzaldehyde, citronellal, trans-cinnamaldehyde or farnesol. In one embodiment, one anti-inflammatory agent is used and is selected from veratraldehyde, piperonyl alcohol, methyl isoeugenol, citronellol, isoamyl cinnamate, ethyl 2-amino benzoate, diethyl malonate, ethyl salicylate, benzaldehyde, citronellal, trans-cinnamaldehyde, farnesol, limonene, linalool, and geraniol.

In one embodiment, the anti-inflammatory agent is veratraldehyde. In one embodiment, the anti-inflammatory agent is piperonyl alcohol. In one embodiment, the anti-inflammatory agent is methyl isoeugenol. In one embodiment, the anti-inflammatory agent is ethyl 2-amino benzoate. In one embodiment, the anti-inflammatory agent is isoamyl cinnamate. In one embodiment, the anti-inflammatory agent is diethyl malonate.

In one embodiment of the invention disclosed herein, methyl isoeugenol is used to treat arthritis, rheumatoid arthritis, osteoarthritis, psoriatic arthritis, spondyloarthritis, SLE or gout. Preferably, methyl isoeugenol is used to treat rheumatoid arthritis or osteoarthritis.

In another embodiment of the invention disclosed herein, veratraldehyde is used to treat arthritis, rheumatoid arthritis, osteoarthritis, psoriatic arthritis, spondyloarthritis, SLE or gout. Preferably, veratraldehyde is used to treat rheumatoid arthritis or osteoarthritis.

In another embodiment of the invention disclosed herein, piperonyl alcohol is used to treat arthritis, rheumatoid arthritis, osteoarthritis, psoriatic arthritis, spondyloarthritis, SLE or gout. Preferably, piperonyl alcohol is used to treat rheumatoid arthritis or osteoarthritis.

Advantageously, agents such as piperonyl alcohol and veratraldehyde have already been approved for use as flavourings in several jurisdictions, so would not require extensive, time-consuming, and costly market authorisations. Table 1 provides a list of compounds that have already been approved for use as additives and flavourings by the EU and FEMA (Food and Extract Manufacturer's Association of the United States) and the FDA (Food and Drug Administration).

TABLE 1

| | | EU | EU | | |
|---|---|---|---|---|---|
| Name | CAS | additive | flavour | FEMA | FDA |
| Piperonyl alcohol | 495-76-1 | No | Yes | No | No |
| Veratraldehyde | 102-14-9 | No | Yes | Yes | Yes |
| Methyl isoeugenol | 93-16-3 | No | Yes | Yes | No |
| Citronellol | 106-22-9 | No | Yes | Yes | Yes |
| Isoamyl cinnamate | 7779-65-9 | No | Yes | Yes | Yes |
| Ethyl 2-amino | 87-25-2 | No | Yes | Yes | Yes |
| benzoate | | | | | |
| Diethyl malonate | 105-53-3 | No | Yes | Yes | Yes |
| Ethyl salicylate | 118-61-6 | No | Yes | Yes | Yes |
| Benzaldehyde | 100-52-7 | No | Yes | Yes | Yes |
| Citronellal | 106-23-0 | No | Yes | Yes | Yes |
| trans- | 104-55-2 | No | Yes | Yes | Yes |
| Cinnamaldehyde | | | | | |
| Farnesol | 4602-84-0 | No | Yes | Yes | Yes |
| Limonene (S) | 5989-54-8 (S) | No | Yes | No | Yes |
| Limonene (R) | 5989-27-5 (R) | No | Yes | Yes | Yes |
| Linalool | 78-70-6 | No | Yes | Yes | Yes |
| Geraniol | 106-24-1 | No | Yes | Yes | Yes |

### The Food

The anti-inflammatory agent may be combined with a food product. The skilled person will be familiar with food products and their typical ingredients. The food product may be a solid or liquid food. In one embodiment the food product is a beverage. In one embodiment, the food product comprises one or more ingredients selected from carbohydrate (such as fibre, starch and/or sugar), protein, fat, vitamins and minerals; or combinations thereof. The food product may comprise water; flavour enhancers, such as sweetener; preservatives; colourants; fish and animal products, such as milk, egg, meat and fat; plant products such as wheat, flour, oats, vegetable, fruit, nuts, seeds, seaweed, herbs, and spices; and fungal products such as mushroom and yeast; or combinations thereof.

Other food additives may be provided such as gelling agents, foaming or anti-foaming agents, and thickeners.

The anti-inflammatory agent may be provided in the food product in a therapeutically effective amount. The anti-inflammatory agent may be provided in the product in the range of 50 mg to 4000 mg and/or in an amount to provide a dosage of 0.7 mg/kg bodyweight to 57.5 mg/kg bodyweight. The anti-inflammatory agent may be provided in the product in an amount to provide a dosage of at least 0.7 mg/kg bodyweight. In another embodiment, the anti-inflammatory agent may be provided in the food product in an amount of less than 4000 mg. Alternatively, the anti-inflammatory agent may be provided in the food product in an amount of more than 4000 mg.

### The Subject

The subject may be an individual in need of an anti-inflammatory agent for use in treating or preventing an inflammatory disorder, such as type 2 diabetes or insulin resistance. In one embodiment, the subject will have type 2 diabetes or has symptoms of type 2 diabetes. In one embodiment the subject will have insulin resistance or will have symptoms of insulin resistance.

In one embodiment the subject is a mammalian subject. In a preferred embodiment, the subject is a human subject. The subject may be an adult human. The subject may be male or female. The subject may be an adult over the age of 18, alternatively, the subject may be a child under the age of 18. Additionally, or alternatively, the subject may be a non-human animal, such as a livestock animal (e.g. cattle, bull, horse, stallion, stag, ox or buffalo), or a domestic pet (e.g. a dog, cat, or equine animal).

In one embodiment the subject will have prediabetes. Prediabetes is diagnosed when a subject has blood glucose levels higher than normal, but not yet high enough for diabetes to be diagnosed.

In one embodiment the subject will be at risk of developing type 2 diabetes or insulin resistance.

In one embodiment the subject will not have either type 2 diabetes or insulin resistance.

In one embodiment the subject may have an inflammatory disease/condition including but not limited to type 1 diabetes, prediabetes, gestational diabetes, metabolic disorders, cardiovascular disease (CVD), connective tissue diseases, arthritis and other joint diseases, periodontitis, gout, spondyloarthritis, uveitis, hidradenitis suppurativa, systemic lupus erythematosus (SLE), chronic obstructive pulmonary disease (COPD), psoriasis, rheumatoid arthritis, psoriatic arthritis, inflammatory bowel disease, liver disease, pain, neurological and psychiatric disorders, neurodegenerative disorders (e.g. Alzheimer's, dementia), depression, stress, narcolepsy, COVID 19, viral infections; or combinations thereof.

In an alternative embodiment the subject may have an inflammatory disease/condition including but not limited to type 2 diabetes, prediabetes, gestational diabetes, metabolic disorders, cardiovascular disease (CVD), connective tissue diseases, arthritis and other joint diseases, psoriatic arthritis, periodontitis, gout, spondyloarthritis, uveitis, hidradenitis suppurativa, systemic lupus erythematosus (SLE), chronic obstructive pulmonary disease (COPD), psoriasis, rheumatoid arthritis, inflammatory bowel disease, liver disease, pain, neurological and psychiatric disorders, neurodegenerative disorders (e.g. Alzheimer's, dementia), depression, stress, narcolepsy, COVID 19, viral infections; or combinations thereof.

The compositions for use according to the claimed invention will comprise a therapeutically effective amount of the anti-inflammatory agent.

The subject may consume the anti-inflammatory agent, for example in food, at least once a day, at least twice a day or at least once a week.

In one embodiment, the anti-inflammatory agent may be formulated into a capsule suitable for oral delivery, such as suitable for consumption as a food supplement.

In one embodiment, the anti-inflammatory agent(s) is not for use as a flavouring.

### OTHER ASPECTS

According to another aspect of the present invention, there is provided a food product for use as a medicament, wherein the food product comprises an anti-inflammatory food additive described herein and a food product.

According to another aspect of the present invention, there is provided a food product for use in treating or preventing an inflammatory disease or condition, such as insulin resistance or Type 2 diabetes, in a subject wherein the food product comprises an anti-inflammatory agent that comprises veratraldehyde, or piperonyl alcohol.

According to another embodiment of the invention, there is provided an encapsulated composition for use in treating or preventing an inflammatory disease or condition, such as type 2 diabetes or insulin resistance, in a subject in need thereof, wherein the encapsulated composition comprises an anti-inflammatory agent described herein.

The encapsulated composition may be suitable for oral consumption. Preferably this encapsulated composition is a pill, such as a supplement pill. The encapsulated composition may be encapsulated in a capsule, such as a dissolvable/digestible polymer capsule. The skilled person will be familiar with capsule types, which are typically made from gelling agents, such as gelatin or plant derivatives such as hydroxypropyl methylcellulose (HPMC). The encapsulated composition may be in the form of a colloidal delivery system, such as encapsulated within microemulsion droplets, emulsion droplets, solid fat particles, liposomes, or microgels.

According to another aspect of the invention, there is provided a method of making a food product for use in treating or preventing an inflammatory disorder, such as type 2 diabetes or insulin resistance, in a subject said method comprising combining an anti-inflammatory agent described herein with a food product.

The food product may comprise one or more of a flavouring, sugar, starch, fibre, protein, milk, juice, vitamin, beverage, cereal, pre-prepared meal, confectionary, snack bar, meal replacement product, or supplement. The food product may comprise meat, fish, fungal or plant extracted material; or combinations thereof.

The inflammatory disease or condition to be treated or prevented may be selected from prediabetes, Type 2 diabetes, insulin resistance, gestational diabetes, metabolic disorders, cardiovascular disease (CVD), connective tissue diseases, arthritis and other joint diseases, periodontitis, gout, spondyloarthritis, uveitis, hidradenitis suppurativa, systemic lupus erythematosus (SLE), chronic obstructive pulmonary disease (COPD), psoriasis, psoriatic arthritis, rheumatoid arthritis, inflammatory bowel disease, liver disease, pain, neurological and psychiatric disorders, neurodegenerative disorders (e.g. Alzheimer's, dementia), depression, stress, narcolepsy, COVID 19, and viral infections; or combinations thereof.

According to another aspect of the present invention, there is provided an anti-inflammatory agent for use in treating or preventing an inflammatory disorder in a subject by inhibiting gene expression, optionally wherein the anti-inflammatory agent is veratraldehyde, or piperonyl alcohol, or methyl isoeugenol.

According to another aspect of the present invention, there is provided a method of treating or preventing an inflammatory disorder in a subject by inhibiting gene expression, the method comprising administering an anti-inflammatory agent, optionally wherein the anti-inflammatory agent is veratraldehyde, or piperonyl alcohol, or methyl isoeugenol.

The inflammatory disorder may be arthritis, rheumatoid arthritis, osteoarthritis, psoriatic arthritis, spondyloarthritis, gout or SLE. The arthritis may be rheumatoid arthritis, osteoarthritis, psoriatic arthritis, or spondyloarthritis. Preferably the inflammatory disorder is rheumatoid arthritis or osteoarthritis.

In one embodiment, there is provided an anti-inflammatory agent for use in treating or preventing arthritis, rheumatoid arthritis, osteoarthritis, psoriatic arthritis, spondyloarthritis, gout or SLE in a subject by inhibiting gene expression, optionally wherein the anti-inflammatory agent is veratraldehyde, or piperonyl alcohol, or methyl isoeugenol.

In one embodiment, a method of treating or preventing arthritis, rheumatoid arthritis, osteoarthritis, psoriatic arthritis, spondyloarthritis, gout or SLE in a subject by inhibiting gene expression, the method comprising administering or consuming an anti-inflammatory agent, optionally wherein the anti-inflammatory agent is veratraldehyde, or piperonyl alcohol, or methyl isoeugenol.

According to another aspect of the present invention, there is provided a method of inhibiting an inflammatory response in a cell by inhibiting gene expression, the method comprising contacting the cell with an anti-inflammatory agent, optionally wherein the anti-inflammatory agent is veratraldehyde, or piperonyl alcohol, or methyl isoeugenol.

The cell may be an immune cell. The cell may be a T-cell, a B-cell, a monocyte, a macrophage or a dendritic cell. Preferably the cell is a macrophage.

Inhibiting gene expression can refer to inhibiting the expression of inflammatory genes that are overexpressed in inflammation or an inflammatory disorder (e.g., il-1, il-6 and/or tnfa). Therefore, inhibiting gene expression can refer to inhibition of il-1, il-6 and/or tnfa, preferably inhibiting tnfa.

According to another aspect of the invention, there is provided the use of an anti-inflammatory agent to suppress expression of genes overexpressed in inflammation or an inflammatory disorder, optionally wherein the anti-inflammatory agent is veratraldehyde, or piperonyl alcohol, or methyl isoeugenol.

Inhibiting gene expression or expression of genes overexpressed in inflammation or an inflammatory disorder can refer to inhibition of il-1, il-6 and/or tnfa, preferably inhibiting tnfa.

The anti-inflammatory agents referred to herein, particularly methyl isoeugenol, veratraldehyde and/or piperonyl alcohol may be used to treat or prevent an inflammatory disorder or inhibit an inflammatory response by inhibiting gene expression.

According to another aspect of the invention, there is provided methyl isoeugenol for use to treat or prevent arthritis in a subject.

According to another aspect of the invention, there is provided a method of treatment of arthritis in a subject, the method comprising the administration of methyl isoeugenol to the subject.

The method may enhance Col2A1 expression and/or collagen production in chondrocytes of the subject. In one embodiment, the enhancement of the Col2A1 expression may be a direct effect of the methyl isoeugenol on Col2A1 expression, for example not mediated by inflammatory cytokines. Alternatively, the enhancement of the Col2A1 expression may be an indirect effect of the methyl isoeugenol on Col2A1 expression, for example mediated by inflammatory cytokines. In another embodiment, the enhancement of Col2A1 may be both a direct and indirect effect.

The use of methyl isoeugenol for treatment or prevention of arthritis may be one or more of rheumatoid arthritis, osteoarthritis and psoriatic arthritis.

The use of methyl isoeugenol for treatment or prevention of arthritis may comprise oral, systemic or local administration. For example, administration may de local injection at the site of the arthritis, such as in a joint. The administration may be by injections into synovial fluid or tissue of the joint. In another embodiment, the local administration may be provided by a controlled release implant, for example at the site of arthritis.

According to another aspect of the invention, there is provided the use of methyl isoeugenol to enhance Col2A1 expression and/or collagen production in chondrocytes.

In one embodiment, methyl isoeugenol is used to enhance Col2A1 expression and/or collagen production in chondrocytes.

The use may be in vitro.

### Definitions

An inflammatory disease or condition may include inflammatory-related disorder, which is understood by those or ordinary skill in the art to be a disorder that it attributable to excessive and/or chronic inflammation in a subject. As used herein, examples of inflammatory diseases/conditions may include prediabetes, Type 2 diabetes, insulin resistance, gestational diabetes, metabolic disorders, cardiovascular disease (CVD), connective tissue diseases, arthritis and other joint diseases, periodontitis, gout, spondyloarthritis, uveitis, hidradenitis suppurativa, systemic lupus erythematosus (SLE), chronic obstructive pulmonary disease (COPD), psoriasis, rheumatoid arthritis, psoriatic arthritis, inflammatory bowel disease, liver disease, pain, neurological and psychiatric disorders, neurodegenerative disorders (e.g. Alzheimer's, dementia), depression, stress, narcolepsy, COVID 19, viral infections.

Type 2 diabetes is defined as a metabolic disorder in which persistent hyperglycaemia (characterised by random plasma glucose more than 11.1 mmol/L, HbA1c of 48 mmol/mol (6.5%) or more, or fasting plasma glucose level of 7.0 mmol/L or more) is caused by insulin resistance and a relative insulin deficiency. The term “type 2 diabetes” encompasses diseases previously referred to as “non-insulin-dependent diabetes mellitus (NIDDM)” and “adult-onset diabetes”. Type 2 diabetes may be considered to be an inflammatory disorder or an inflammatory-related disorder.

Insulin resistance is defined as a condition when cells no longer respond sufficiently to insulin which affects glucose uptake.

Type 1 diabetes is defined as an autoimmune disease that causes insulin producing beta cells in the pancreas to be destroyed, preventing the body from being able to produce enough insulin to regulate blood glucose levels. The term type 1 diabetes encompasses diseases previously referred to as “insulin dependent diabetes” or “juvenile diabetes”.

Gestational diabetes is defined as any degree of glucose intolerance with onset or first recognition during pregnancy. Gestational diabetes is caused when the body cannot make the extra insulin needed during pregnancy.

The term “metabolic disorders” encompasses a group of diseases including but not limited to: familial hypercholesterolemia, Gaucher disease, Hunter syndrome, Krabbe disease, metachromatic leukodystrophy, Niemann-Pick, Phenylketonuria, Porphyria, Tay-Sachs disease and Wilson's disease. Typically, metabolic disorders are the result of a genetic defect that disrupts metabolism.

The term “cardiovascular disease (CVD)” encompasses all heart and circulatory diseases including but not limited to coronary heart disease, angina, heart attack, congenital heart disease, hypertension, stroke and vascular dementia.

The term “connective tissue disease” encompasses diseases including but not limited to rheumatoid arthritis, scleroderma, granulomatosis with polyangiitis, Churg-Strauss syndrome, systemic lupus erythematosus, microscopic polyangiitis or polymyositis/dermatomyositis.

The term “arthritis” encompasses diseases including but not limited to osteoarthritis, rheumatoid arthritis, ankylosing spondylitis, cervical spondylosis, fibromyalgia, systemic lupus erythematosus, gout, psoriatic arthritis, enteropathic arthritis, reactive arthritis, secondary arthritis or polymyalgia rheumatica.

Periodontitis refers to a gum infection. As used herein, the term “periodontitis” also encompasses gingivitis.

Gout refers to a disease that is a type of arthritis caused by the accumulation of urate crystals in a joint.

Spondyloarthritis encompasses a variety of arthritic diseases including but not limited to diseases that affect the back, pelvis, neck and larger joints as well as internal organs.

Spondyloarthritis is characterised by inflammation where ligaments and tendons attach to bones.

Uveitis is a form of eye inflammation that affects the middle layer of tissue in the eye wall (uvea).

Hidradenitis suppurativa is a skin condition that can cause abscesses and scarring of the skin.

Systemic Lupus Erythematosus (SLE) is an autoimmune disease that can affect the joints, skin, brain, lungs, kidneys and blood vessels.

The term “Chronic obstructive pulmonary disease (COPD)” encompasses several lung conditions that cause breathing difficulties including but not limited to emphysema and chronic bronchitis.

Psoriasis refers to a long-term chronic inflammatory skin condition. Rheumatoid arthritis is a chronic inflammatory condition that can affect a wide range of tissues. The term “Inflammatory bowel disease” encompasses inflammatory diseases including but not limited to ulcerative colitis and Crohn's disease.

Liver disease could refer to a number of diseases affecting the liver and that have an immune component, for example autoimmune hepatitis.

The term “pain” as used herein could refer to any pain caused by inflammation.

The term “Neurological and psychiatric disorders” as used herein refers to any neurological or psychiatric disorder that involves inflammation, for example: stroke, Parkinson's disease, Alzheimer's disease, amyotrophic lateral sclerosis, major depression, and schizophrenia

The term “Neurodegenerative disorders” as used herein refers to any neurodegenerative disease that has an inflammatory component, for example: Alzheimer's Disease, Parkinson's Disease, Multiple Sclerosis, and Huntingdon's Disease.

Depression is characterised by persistent feelings of sadness and a loss of interest in activities, there are several types of depression including but not limited to major depression, persistent depressive disorder, bipolar disorder, seasonal affective disorder, psychotic depression postpartum depression, and premenstrual dysphoric disorder.

Stress is characterised as the feeling of being overwhelmed or unable to cope with mental or emotional pressure.

Narcolepsy has also been found to have an inflammatory component, and so the present invention may be used to provide benefit to a subject with narcolepsy.

COVID-19 refers to disease caused by the virus Sars-Cov-2 including all variants for example the alpha variant, the beta variant, the gamma variant, and the delta variant.

As used herein, the term “viral infections” encompass all disease caused by viruses.

The term “food additive” as used herein is understood to mean an agent that can be included in compounds or compositions fit for consumption by a subject (preferably a human and/or animal subject). The compound or composition may be a food product, or alternatively, the compound or composition fit for consumption may be a medicine or pharmaceutical product. The compound or composition may be comprised in a solid food product, a liquid food product, a drink, a powder, a gel an encapsulated tablet, or as an unencapsulated tablet. The term “anti-inflammatory agent” or “agent” used herein may be used interchangeably with “anti-inflammatory food additive”, or “food additive”.

The term “anti-inflammatory food additive” used herein is understood to mean a food additive which has an anti-inflammatory effect in a subject such as reducing the level of Tumour Necrosis Factor (TNF) and/or interleukin-1 (IL-1) in the subject. The anti-inflammatory effect in the subject may comprise the reduction in the level of Tumour Necrosis Factor (TNF) and/or interleukin-1 (IL-1) from an elevated level in the subject. The elevated level may be a level that is higher than an average healthy individual that is not suffering from or not considered to be at risk of suffering from insulin resistance or type 2 diabetes. Elevated levels of TNF or IL-1 may be measured by any means known in the art, for example by ELISA. Elevated levels of TNF/IL-2 may be determined by comparing a test sample to a control sample.

The reduction in the level Tumour Necrosis Factor (TNF) and/or interleukin-1 (IL-1) in the subject may be a therapeutically effective level of reduction. In one embodiment, the reduction in the level Tumour Necrosis Factor (TNF) and/or interleukin-1 (IL-1) in the subject may be at least a 10% or 20% reduction. The level of Tumour Necrosis Factor (TNF) and/or interleukin-1 (IL-1) may refer to the circulating serum level.

The term “food product” as used herein is understood to mean any product (solid or liquid) that is suitable and intended for consumption by a subject. The food product may be consumed for nutrition or pleasure by the subject. For example, a food product may be a composition that can be consumed in reasonable quantities without negative health consequences. A food product may be a flavouring, sugar, starch, fibre, protein, milk, juice, vitamin, beverage, cereal, pre-prepared meal, confectionary, snack bar, meal replacement product, or supplement. The food product may be for use in medicine or a pharmaceutical composition.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

### FIGURES

FIG. 1: Male mice fed ad libitum a chow diet (18% fat, Lean, n=15) or high-fat diet (HFD) (60% fat), starting at 2 months of age for all groups and continued for 12 weeks. From 8-12 weeks HFD groups were separated into H2O alone group (n=25), H2O+1% w/v compound groups (n=13-20) and H2O+0.5% w/v compound groups (n=5). (A) Glucose-tolerance test (GTT) for compounds from 0-120 min and (B) the total area under the curve (AUC) analysis for 1% w/v compounds from 0-120 min. (C) (Left) Glucose levels at 0 min and (right) glucose levels at 120 min only. All data are presented as the mean±SEM of 4 in vivo experiments carried out on different days and were subjected to unpaired Student's t test (*p<0.5, **p<0.01, ***p<0.001, ****p<0.0001).

FIG. 2: Male mice fed ad libitum a chow diet (18% fat, Lean, n=10) or high-fat diet (HFD) (60% fat), starting at 2 months of age for all groups and continued for 12 weeks. From 8-12 weeks HFD groups were separated into H2O alone group (n=25) or H2O+compound groups, which were added to drinking water at 1% w/v (n=13-20, Veratraldehyde and Piperonyl Alcohol) or 0.5% w/v of each compound when in combination (n=5). (A) Glucose-tolerance test (GTT) up to 120 min. (B) Glucose levels at timepoint 0 min only. (C) The total area under the curve (AUC) analysis for GTT up to 120 min. All data are presented as the mean±SEM of in vivo experiments carried out on different days and were subjected to unpaired Student's t test (**p<0.01, ****p<0.0001).

FIG. 3: ELISA analysis of TNF expression in media from cells pretreated with DMSO (vehicle) or Veratraldehyde for 2 hrs and stimulated with LPS for 24 hrs. (A) human THP1 cells treated with Veratraldehyde (200 μM, 100 μM and 50 μM) and LPS (1 mg/ml). (B) human peripheral blood mononuclear cell (PBMC) treated with Veratraldehyde (100 μM) and LPS (100 μg/ml). (C) murine bone-marrow-derived macrophages (BMDMs) isolated from C57BL/6 WT mice treated with Veratraldehyde (100 μM) and LPS (100 μg/ml). (D) Quantitative RT-PCR of mRNA expression of Tnf in PBMCs pretreated with DMSO (vehicle) or veratraldehyde (100 μM) for 2 hrs and stimulated with LPS (100 μg/ml) for 3 hrs. (E and G) TNF was measured from canine macrophage-like cells (DH82) treated with veratraldehyde (1 mM, 500 uM, 200 uM, 100 uM, 50 uM, 10 uM) and LPS for 24 hours by ELISA. (F) Quantitative RT-PCR of mRNA expression of Tnf in DH82 cells pretreated with DMSO (vehicle) or veratraldehyde (500 μM) for 2 hrs and stimulated with LPS (100 μg/ml) for 1.5 and 3 hrs. (H) DH82 cells were stimulated with DMSO (vehicle) or veratraldehyde (500 μM) for 2 hrs and stimulated with LPS (100 μg/ml) for 3 hours. Cells were washed and media replaced for 24 hours. DH82 media was then transferred to canine chondrocytes (CNC) for 24 hours. COL2A1 mRNA expression was measured by RT-PCR. All data are presented as the mean±SEM of at least three independent experiments and were subjected to paired Student's t test (*p<0.5, **p<0.01, ***p<0.001).

FIG. 4: ELISA analysis of TNF expression in media from cells pretreated with DMSO (vehicle) or Methyl Isoeugenol for 2 hrs and stimulated with LPS for 24 hrs. (A) human THP1 cells treated with Methyl Isoeugenol (200 μM, 100 μM and 50 μM) and LPS (1 mg/ml). (B) human peripheral blood mononuclear cell (PBMC) treated with Methyl Isoeugenol (100 μM) and LPS (100 μg/ml). (C) murine bone-marrow-derived macrophages (BMDMs) isolated from C57BL/6 WT mice treated with Methyl Isoeugenol (100 μM) and LPS (100 μg/ml). (D) Quantitative RT-PCR of mRNA expression of Tnf in PBMCs pretreated with DMSO (vehicle) or Methyl Isoeugenol (100 μM) for 2 hrs and stimulated with LPS (100 μg/ml) for 24 hrs. (E) Quantitative RT-PCR of mRNA expression of COL2A1 in Canine chondrocytes (CNC) pretreated with DMSO (vehicle), Methyl Isoeugenol, NaOH, Vertic Acid, Folic Acid, Piperonyl Alcohol, Veratraldehyde (500 μM) for 24 hrs. All data are presented as the mean±SEM of at least three independent experiments and were subjected to paired Student's t test (*p<0.5, **p<0.01).

FIG. 5: (A) Immunoblot analysis of phosphorylated (p-)JNK and β-Actin in cell lysates from human peripheral blood mononuclear cell (PBMC). Cells were pretreated with DMSO (NT vehicle), veratraldehyde (100 μM) or Methyl Isoeugenol (100 μM) for 2 hours, followed by stimulation with LPS (100 μg/ml) for 1 hour. (B) p-JNK bands were quantified relative to R-Actin using image j software. The value of compound treated wells were compared to vehicle alone.

FIG. 6: ELISA analysis of TNF expression in media from cells pretreated with DMSO (vehicle) or Piperonyl Alcohol for 2 hrs and stimulated with LPS for 24 hrs. (A) human THP1 cells treated with Piperonyl Alcohol (1 mM, 100 μM and 10 μM) and LPS (1 mg/ml). (B) human peripheral blood mononuclear cell (PBMC) treated with Piperonyl Alcohol (100 μM) and LPS (100 μg/ml). (C) Quantitative RT-PCR of mRNA expression of Tnf in PBMCs pretreated with DMSO (vehicle) or Piperonyl Alcohol (100 μM) for 2 hrs and stimulated with LPS (100 μg/ml) for 24 hrs. All data are presented as the mean±SEM of at least three independent experiments.

FIG. 7: ELISA analysis of IL-10 expression in media from cells pretreated with DMSO (vehicle) or (A) citronellol, (B) isoamyl cinnamate, (C) ethyl 2-aminobenzoate, (D) Farnesol (E) diethyl malonate or (F) ethyl salicylate for 2 hrs and stimulated with LPS for 24 hrs. Human THP1 cells treated with compounds at (1 mM, 100 uM and 10 uM) and LPS (1 mg/ml). All data are presented as the mean±SEM of three or four independent experiments and were subjected to paired Student's t test (*p<0.5, **p<0.01).

FIG. 8: ELISA analysis of TNF expression in media from cells pretreated with DMSO (vehicle) or (A) benzaldehyde, (B) citronellal and (C) trans-cinnamaldehyde for 2 hrs and stimulated with LPS for 24 hrs. Human THP1 cells treated with compounds at (1 mM, 100 μM and 10 μM) and LPS (1 mg/ml). All data are presented as the mean±SEM of three or four independent experiments and were subjected to paired Student's t test (*p<0.5, **p<0.01).

FIG. 9: ELISA analysis of IL-10 and TNF expression in media from cells pretreated with DMSO (vehicle) or (A/B) limonene, (C/D) linalool and (E/F) geraniol for 2 hrs and stimulated with LPS for 24 hrs. Human THP1 cells treated with compounds at (1 mM, 100 μM and 10 μM) and LPS (1 mg/ml). All data are presented as the mean±SEM of three or four independent experiments and were subjected to paired Student's t test (*p<0.5).

FIG. 10: ELISA analysis of IL-10 and TNF expression in media from cells pretreated with DMSO (vehicle) or (A/B) piperonyl acetate and (C/D) piperonyl isobutyrate for 2 hrs and stimulated with LPS for 24 hrs. Human THP1 cells treated with compounds at (1 mM, 100 μM and 10 μM) and LPS (1 mg/ml). All data are presented as the mean±SEM of three or four independent experiments and were subjected to paired Student's t test.

FIG. 11: ELISA analysis of IL-10 and TNF expression in media from cells pretreated with DMSO (vehicle) or (A/B) vanillin, (C/D) ethyl vanillin and (E/F) divanillin for 2 hrs and stimulated with LPS for 24 hrs. Human THP1 cells treated with compounds at (1 mM, 100 μM and 10 μM) and LPS (1 mg/ml). (G) DH82 cells were treated with veratric acid (1 mM, 500 uM, 250 uM, 125 uM, 60 uM, 30 uM) for 2 hrs and stimulated with LPS for 24 hrs. All data are presented as the mean±SEM of three or four independent experiments and were subjected to paired Student's t test. The data in FIG. 11 does not show an anti-inflammatory effect despite some literature reports suggesting an anti-inflammatory effect.

FIG. 12: ELISA analysis of IL-10 and TNF expression in media from cells pretreated with DMSO (vehicle) or (A/B) isoeugenyl acetate and (C/D) ethyl cinnamate for 2 hrs and stimulated with LPS for 24 hrs. Human THP1 cells treated with compounds at (1 mM, 100 μM and 10 μM) and LPS (1 mg/ml). All data are presented as the mean±SEM of three or four independent experiments and were subjected to paired Student's t test (*p<0.5).

FIG. 13: (1) DH82 cells are plated and stimulated with the compound, followed by LPS. (2) After 24 hours, TNF has been produced by the cells and (3) This liquid supernatant is removed from the cells and reserved. (4) Chondrocytes are plated and (5) the reserved liquid supernatant containing TNF and other inflammatory cytokines from the DH82 cells is added on top of these cells, thus exposing the cartilage cells to an inflammatory environment, simulating an inflammatory joint. (6) After 24 hours gene expression of COL2A1 is measured.

FIG. 14: (A) DH82 cells were stimulated with DMSO (vehicle) or veratraldehyde (500 μM) for 2 hrs and stimulated with LPS (100 μg/ml) for 3 hours. Cells were washed and media replaced for 24 hours. DH82 media was then transferred to canine chondrocytes (CNC) for 24 hours. COL2A1 mRNA expression was measured by RT-PCR. (B) Quantitative RT-PCR of mRNA expression of COL2A1 in Canine chondrocytes (CNC) pretreated with DMSO (vehicle), Methyl Isoeugenol, NaOH, Vertic Acid, Folic Acid, Piperonyl Alcohol, Veratraldehyde (500 μM) for 24 hrs. All data are presented as the mean±SEM of at least three independent experiments and were subjected to paired Student's t test (*p<0.5).

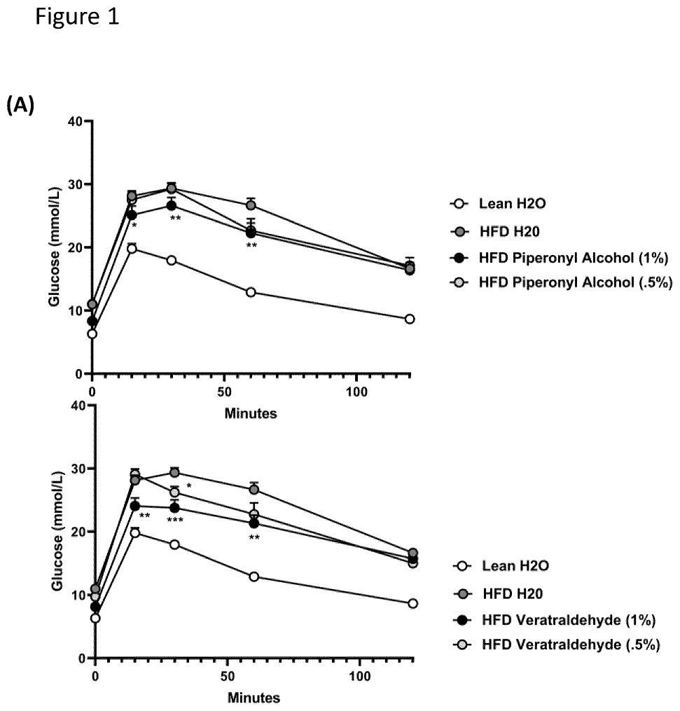

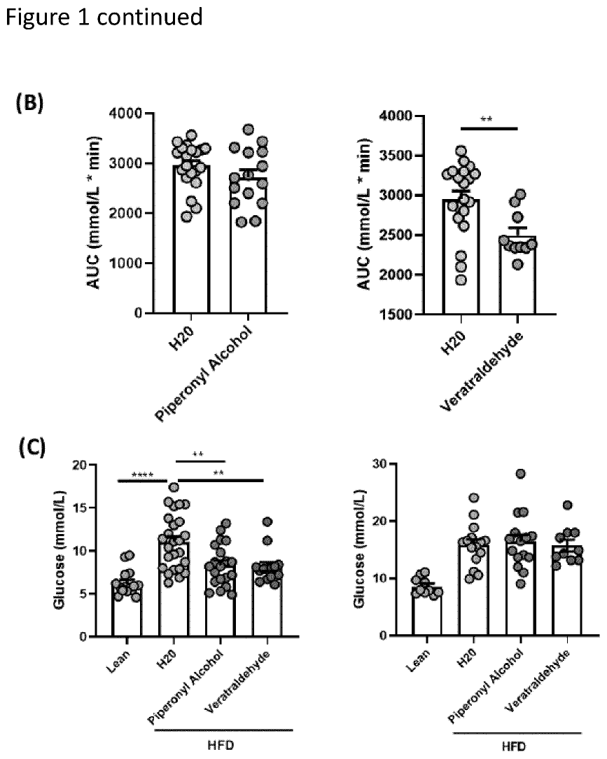

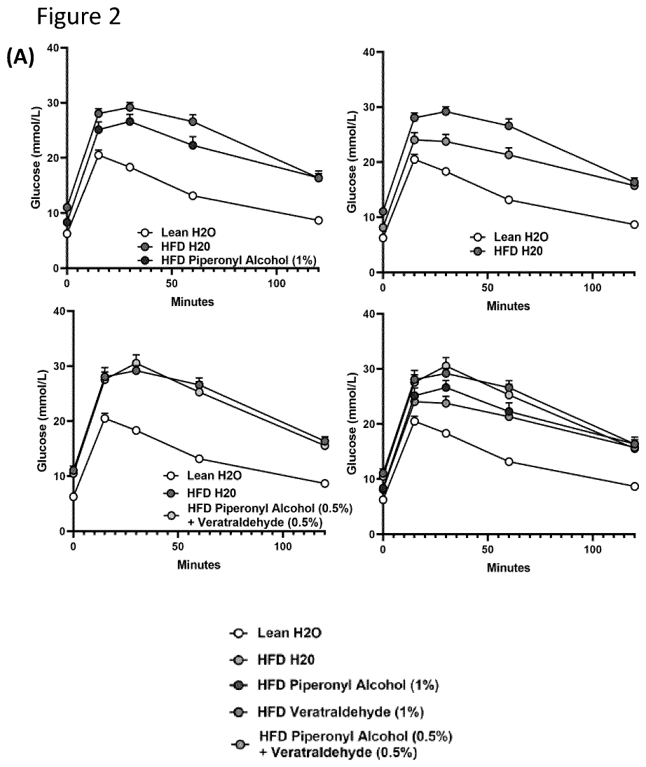

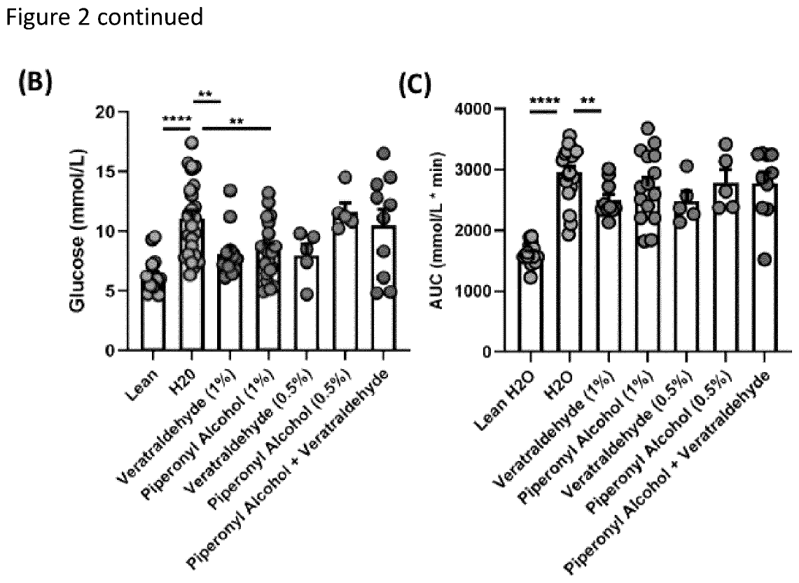

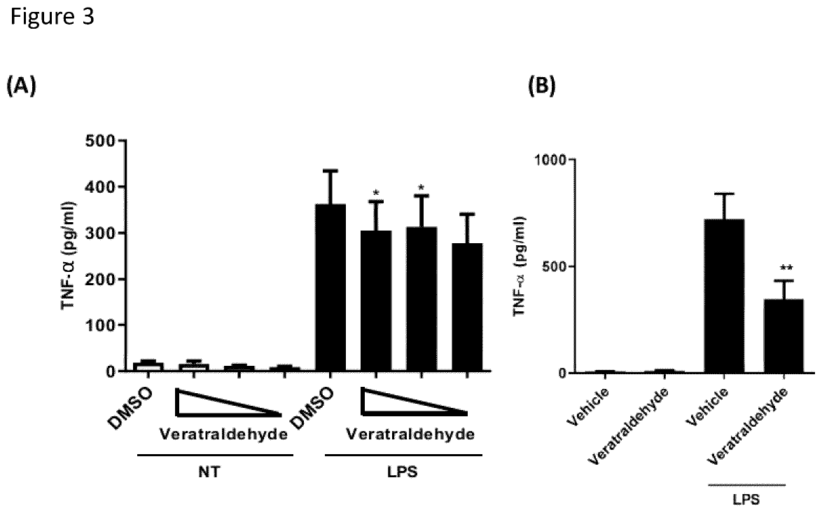

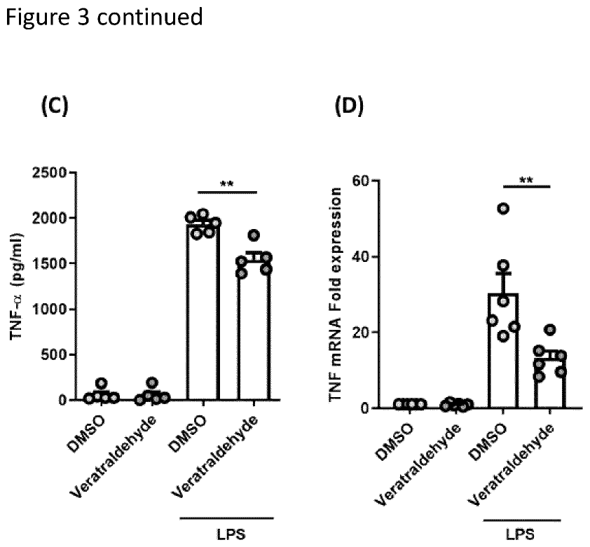

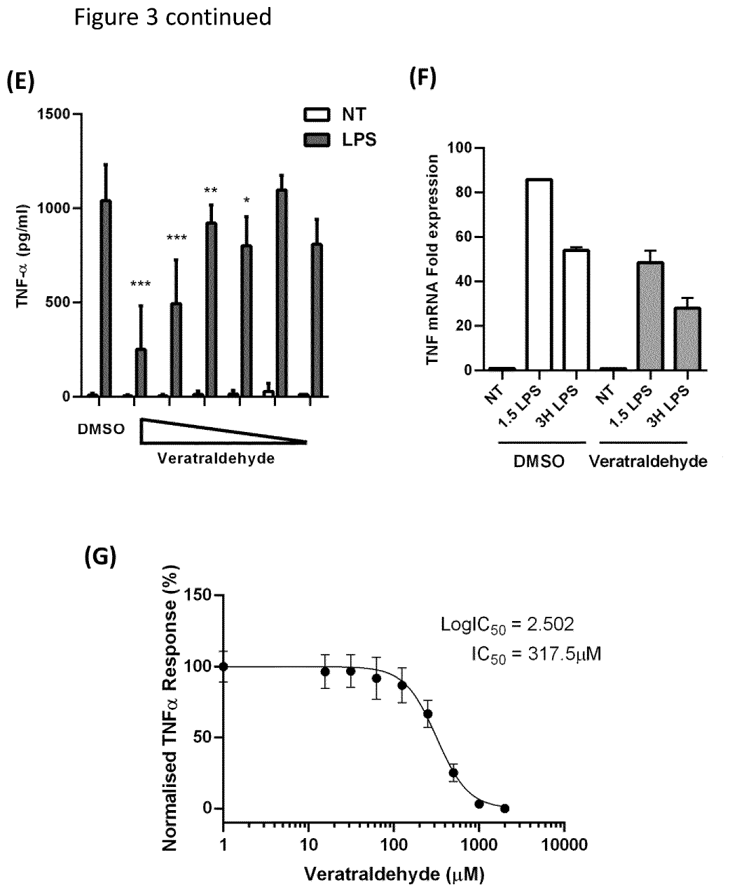

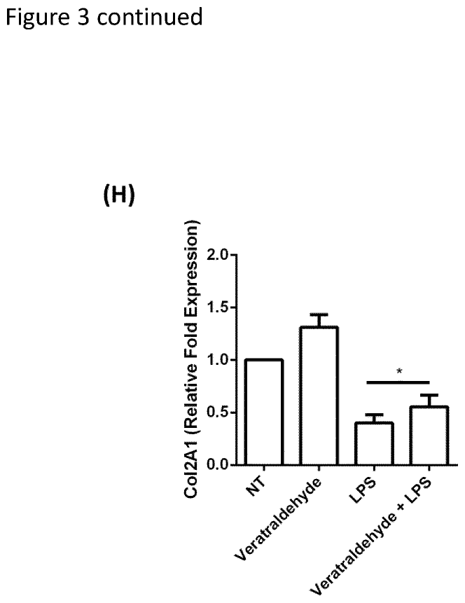

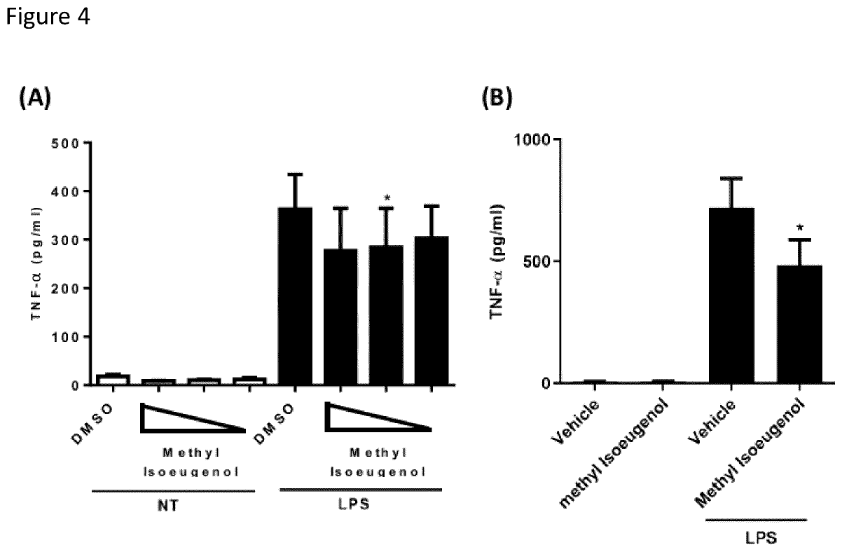

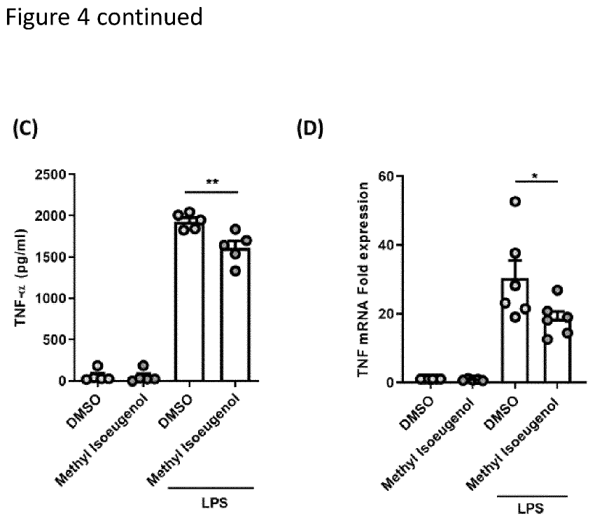

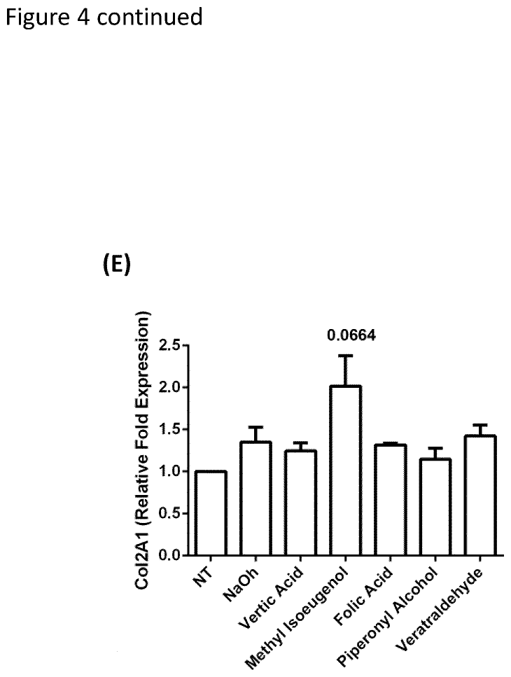

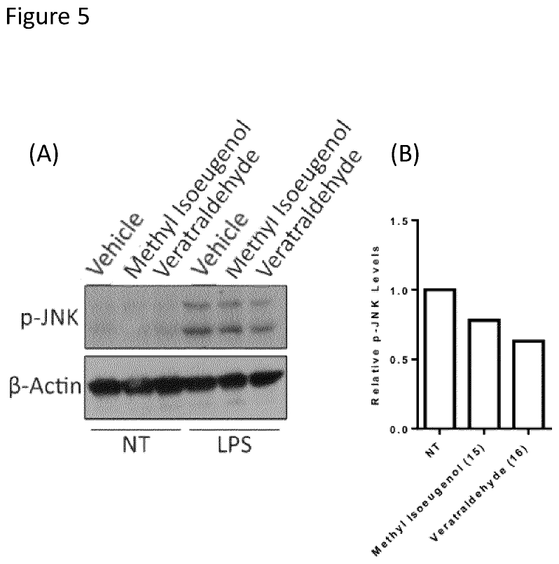

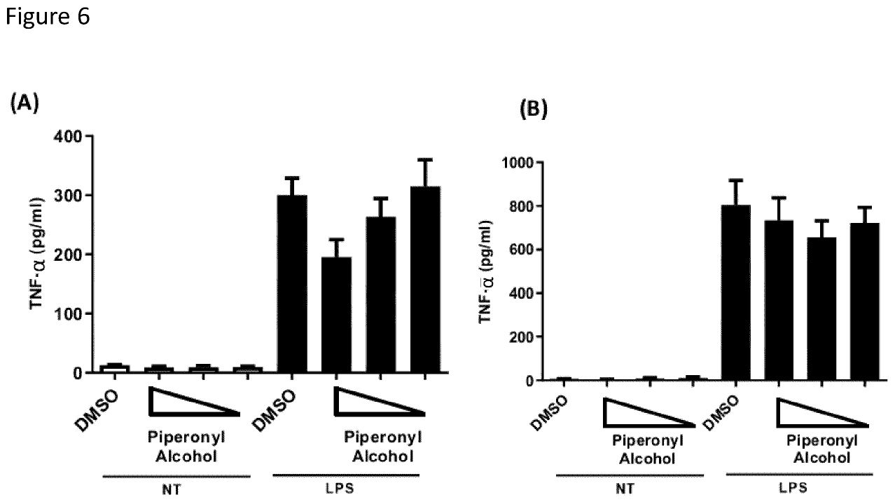

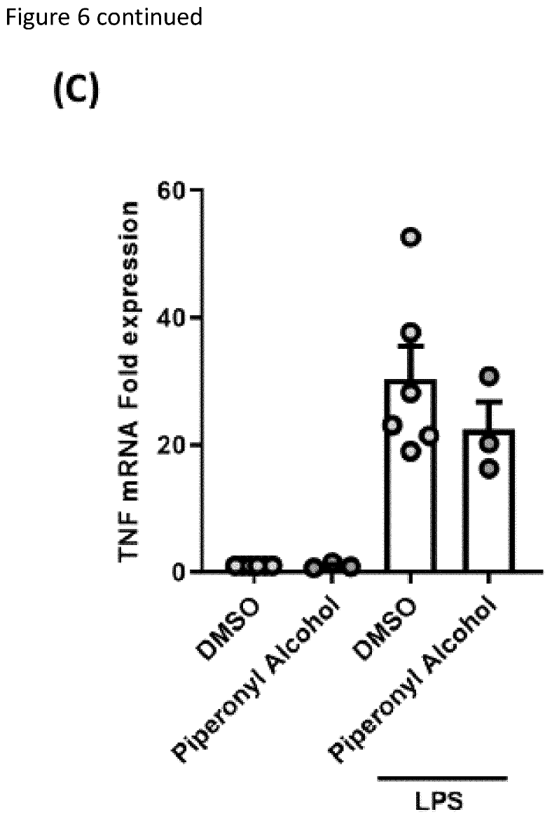

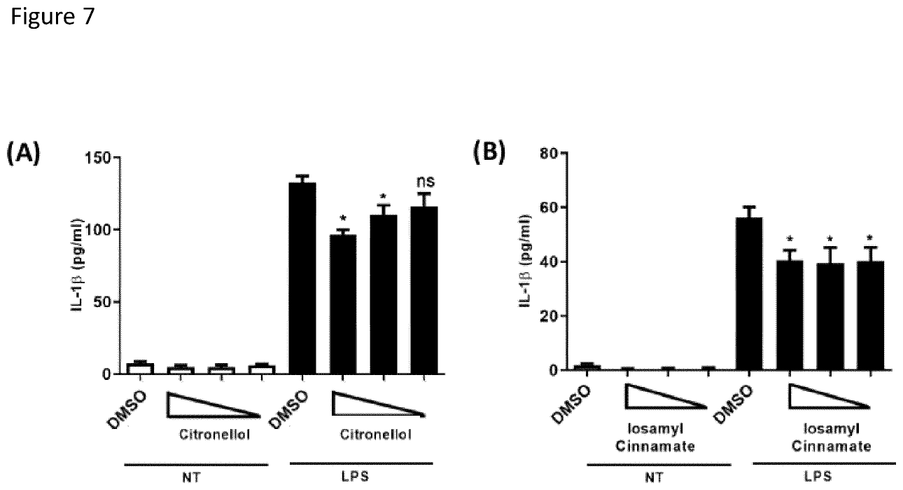

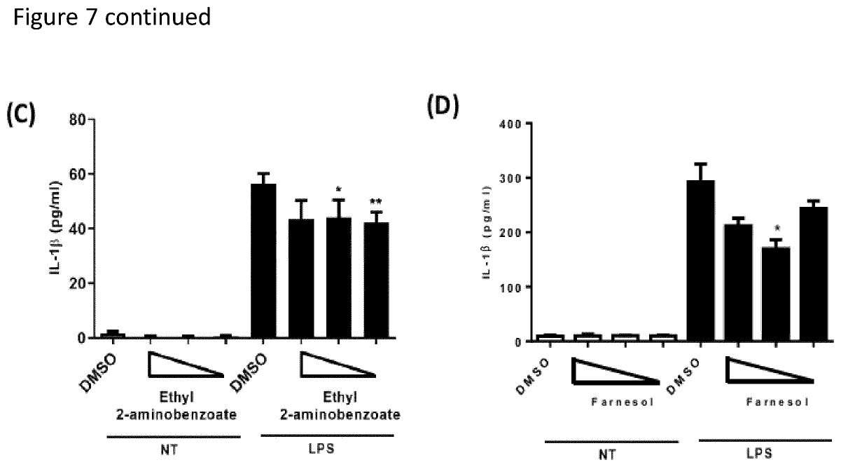

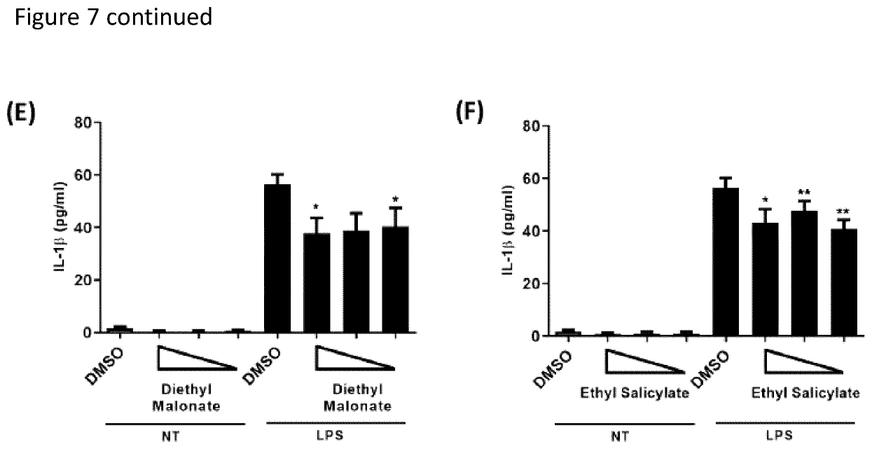

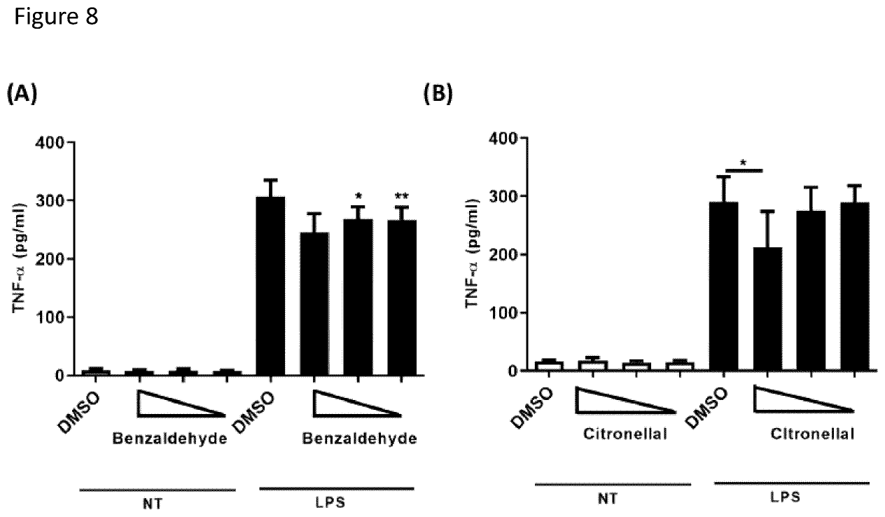

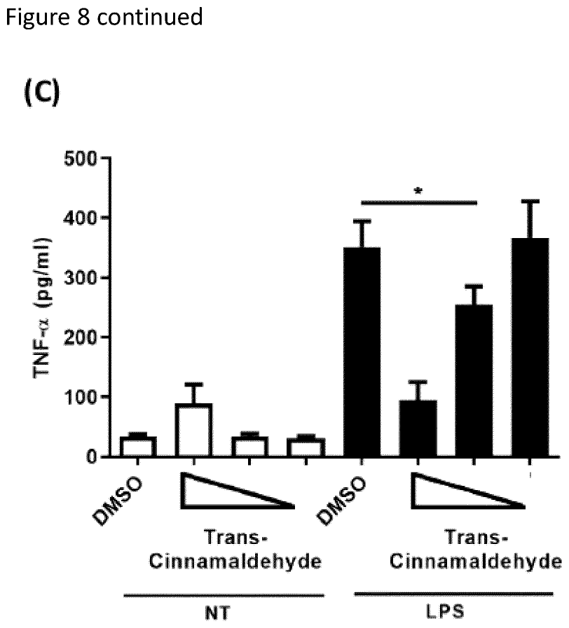

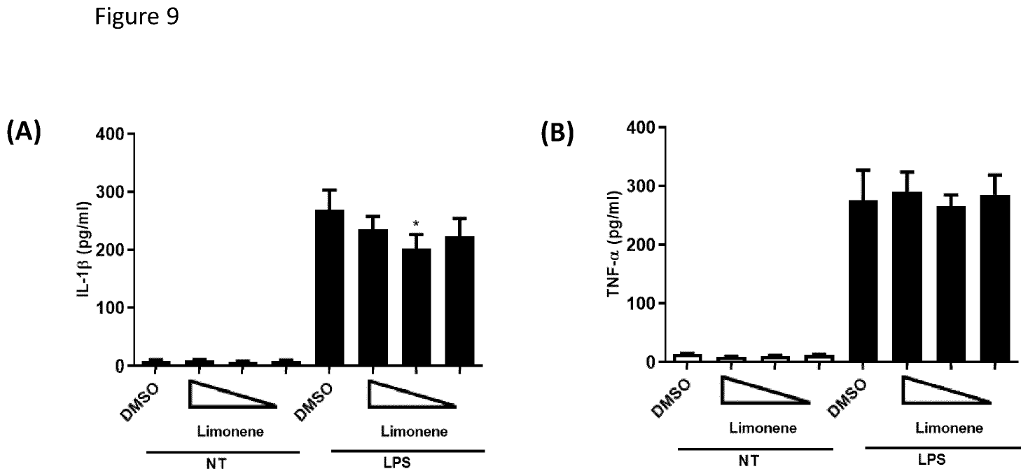

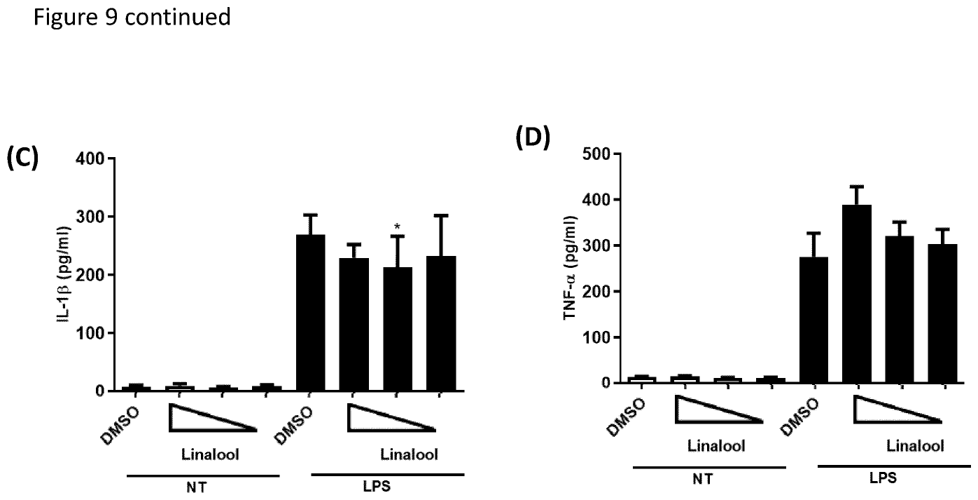

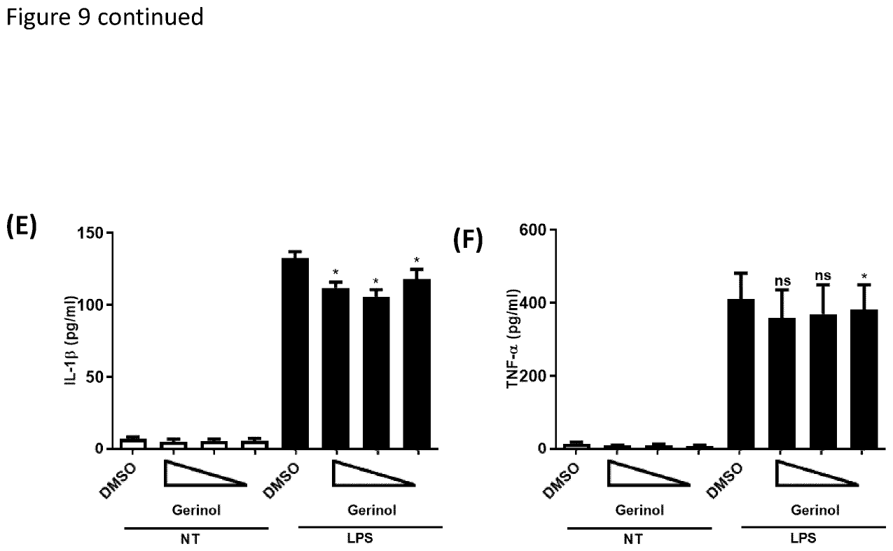

### EXAMPLES

### Introduction

Inflammation is a critically important mechanism for host protection to infection, and resolution of tissue damage, and must be very tightly regulated. Our immune cells express receptors on their surface, allowing them to monitor their surroundings and continuously test for foreign entities and signs of danger. Upon sensing danger, in the form of tissue damage, environmental stress or invading pathogens, the immune system can initiate a signalling cascade which ultimately results in the production of inflammatory mediators. Inflammatory mediators such as TNFα and IL-1 can directly act on the source of the danger, and in parallel recruit and activate other immune subsets. The inflammatory process can drive major damage and destruction so is tightly regulated, so once the danger signal is lost the inflammation resolves.

However, there are numerous conditions associated with uncontrolled inflammation, which fails to resolve, resulting in tissue damage and system dysregulation. This is termed chronic inflammation and is usually in the absence of pathogenic infection, hence called auto-inflammation. Chronic inflammation has been shown to underpin numerous immune diseases including connective tissue diseases (e.g. rheumatoid arthritis, systemic lupus erythematosus, ankylosing spondylitis) metabolic disorders (e.g. type 2 diabetes and cardiovascular disease), neurodegenerative diseases (Alzheimer's) and many cancers.

Our goal has been to identify bioactive compounds capable of affecting the molecular signalling underpinning discrete inflammatory networks, thus allowing us to limit inflammation in situations where excessive inflammation is unwanted. Importantly, our goal is to partially limit the inflammatory process, instead of completely abrogating it, which would potentially result in an increased likelihood of infection and malignancy.

Using a bottom-up approach allowed us to characterise effective compounds to influence inflammation, which are already approved for human consumption.

### Methods

### Culture of THP-1

THP-1 cells were cultured in RPMI supplemented with 10% (v/v) heat inactivated FBS, penicillin G (100 μg/ml) and streptomycin (100 μg/ml) in a 37° C. humidified chamber under a 5% CO2 atmosphere. Cell morphology and confluence, including media acidification colour change were monitored daily. Twice a week, cells were passaged by removal from flask and centrifugation at 400 G. Cells were cultured for a maximum of 25 passages in order to avoid excessive genetic drift. Cells were seeded at 1×10\hat{ }6/ml.

### Culture of HSkMC

HSkMC cells were cultured in Complete growth media in a 37° C. humidified chamber under a 5% CO2 atmosphere. Cell morphology and confluence were monitored daily. Once cells reached approximately 80% confluence, adherent cells were passaged using a 1% (w/v) Trypsin/EDTA solution in PBS. Cells were cultured for a maximum of 8 passages as their growth was limited after this point. Cells were seeded in basal skeletal media in order to remove glucose. Cells were seeded at 5×10⁴/ml for experiments, and 750,000 per T175 in 40 ml for further culturing.

### Culture of DH82

DH82 cells were cultured in EMEM supplemented with 10% (v/v) heat inactivated FBS, penicillin G (100 μg/ml) and streptomycin (100 μg/ml) in a 37° C. humidified chamber under a 5% CO2 atmosphere. Cell morphology and confluence were monitored daily. Once cells reached approximately 80% confluence, adherent cells were passaged using a 1% (w/v) Trypsin/EDTA solution in PBS. Cells were seeded at 4.5×10\hat{ }5/ml.

### Culture of CNC

Canine chondrocyte (CNC) cells were cultured in complete CNC media in a 37° C. humidified chamber under a 5% CO2 atmosphere. Cell morphology and confluence were monitored daily. Once cells reached approximately 80% confluence, adherent cells were passaged using a 1% (w/v) Trypsin/EDTA solution in PBS. Cells were seeded at 1×10\hat{ }5/ml.

### Generation and Culture of Murine Bone Marrow Cells

WT mice aged 8-12 weeks were sacrificed by cervical dislocation. Mice were first sterilised with 70% EtOH before removal of the femur and tibia under aseptic conditions in a laminar flow hood. Bone marrow was removed by flushing the bones with a 274 gauge needle containing 20 ml PBS and cells were centrifuged again at 400 g for 5 min. Pellets were re-suspended in 10 ml DMEM and cells were counted using a haemocytometer. Cells were seeded at 2×10⁶ cells/ml and rested for 2 hours prior to stimulation.

### Total Cellular RNA Extraction

To avoid contamination of RNA samples, all reagents and labware used were certified as RNase free. Gloves were worn and changed frequently in order to avoid introducing RNase contamination. All surfaces were decontaminated with RNase Zap.

BMDM, THP and PBMC cells were seeded at 1×10⁶ cells/ml with 3 ml media in 6-well plates for all mRNA analysis experiments. Harvesting for total RNA was carried out by first washing with PBS, scraping the cells, and centrifuging at 6,500 g for 5 minutes at 4° C. Cell pellets were re-suspended with 500 μl of Trizol and incubated at RT for 5 minutes to ensure full homogenisation. 150 μl of chloroform was added and the mixture was first inverted and then vortexed for 5 sec. Samples were incubated at RT for 10 minutes, during which time, phase separation begins. This is completed by centrifugation at 12,000 g for 15 minutes at 4° C. 150 μl of the upper aqueous phase was transferred to a new sterile tube, avoiding contamination with the red phenol-chloroform phase or the interphase. 150 μl of isopropanol was added to the aqueous phase and the mixture was first inverted and then vortexed for 5 sec. Samples were incubated at RT for 5 minutes, followed by centrifugation at 12,000 g for 15 minutes at 4° C. The isopropanol was carefully removed without disturbing the precipitated RNA pellet. 900 μl of 75% (v/v) EtOH was added to the pellet and vortexed briefly. Samples were centrifuged at 6,500 g for 5 min at 4° C., after which the ethanol was removed and the RNA pellets were air dried in a laminar flow hood for 10 minutes. The RNA pellet was re-suspended in 20 μl of RNase free water and incubated at 60° C. for 10 minutes. A Nanodrop™ spectrophotometer was used to quantify the concentration of RNA in each sample. The ratio of the absorbance at 260 and 280 nm was used to assess the purity of the RNA sample, where a ratio of 1.8-2.0 indicated pure RNA. RNA samples were stored at −80° C.

First Strand cDNA Synthesis from Total Cellular RNA

Complementary DNA (cDNA) is a DNA copy synthesised from mRNA. This reaction was carried out in a sterile microcentrifuge tube. 2 μg of quantified total cellular RNA was diluted in nuclease-free water to a final volume of 12 μl. 1 μl of random primers (0.5 μg) was added to the sample and incubated at 70° C. for 5 min, followed by incubation of the sample on ice. The random primers consist of a mixture of hexamers and anchored-dT primers which results in an even coverage of the RNA template. The following master premix was prepared:

| RNA | 500 | ng |
|---|---|---|
| H₂O | Variable | |
| Qscript 5X Buffer | 4 | μl |
| Qscript Reverse Transcriptase | 1 | μl |

Total Volume 20 ul

After addition of the premix to the sample, mixtures were incubated at 25° C. to facilitate primer annealing. The mixture was incubated at 42° C. for 45 min. The reaction was then terminated by incubation at 85° C. for 5 min, followed by chilling on ice. cDNA samples were stored at −20° C. or immediately amplified by PCR.

### Real-Time DNA Amplification by PCR

Previously generated cDNA were subjected to quantitative real-time PCR analysis, allowing for a comparative quantification between various DNA samples during the PCR amplification process. Specifically designed primers correlating to a targeted region of interest were used. The reagents of the reaction premix described below were added on ice.

| | | |
|---|---|---|
| Perfecta SYBR Green ROX premix | 10 | μl |
| Sense Primers | 2.5 | μl |
| Anti-Sense Primers | 2.5 | μl |
| H₂O | 3 | μl |

18 μl of the above reaction premix was added to an Applied Biosystems 96-well plate, followed by 2 μl of previously prepared template DNA. The PCR was carried out using an Applied Biosystems Step One™ real-time PCR instrument. Samples were first incubated at 95° C. for 5 minutes to facilitate denaturation of template DNA. Samples were then subjected to 30 cycles of first 95° C. for 30 sec, followed by 57° C. for 30 sec and 72° C. for 45 sec. Each stage of this cycling step encompassed denaturation, annealing and extension of the product, respectively. SYBR green binds to double stranded DNA product during the annealing and extension stage, which after absorbance of 497 nm blue light, can be measured by emitted 520 nm green light. The cycling step was followed by a melt curve analysis, which requires the products to be heated from 60° C. to 90° C. One peak on the melt curve verified the presence of a single amplification product. Relative quantification of the targeted gene expression activity was determined using the Crossing Threshold (CT) method. The generated CT value reflected the target well exceeding a preprogramed fluorescence threshold, indicating the presence of a sufficient number of the target amplicons. The cycle which the CT value was reached was recorded and ΔCT value could be determined by subtracting the control gene CT value from the targets. The relative fold change of the genes was further determined using 2-ΔCT.

### Intracellular Protein Extraction

To avoid protein degradation, all equipment was pre-chilled and all steps in the extraction process were carried out on ice unless otherwise stated. To generate intracellular protein extracts, adherent cells (after removal of media), were washed gently with PBS and then scraped in 1 ml PBS and transferred to fresh 1.5 ml tubes. Samples were centrifuged at 6,500 g for 10 minutes at 4° C., after which the PBS was removed. In the case of non-adherent cells, the media containing non-adherent cells was removed and added to a 15 ml tube. Cells were scraped in 1 ml PBS and transferred to the same 15 ml tube. Samples were centrifuged at 6,500 g for 10 minutes at 4° C., after which the PBS was removed. Cells were re-suspended in 1 ml PBS, transferred to a new 1.5 ml tube and once again centrifuged at 6,500 g for 10 minutes at 4° C. and the PBS was removed. In the case of both adherent and semi-adherent cells, cellular pellets were re-suspended in 90 μl lysis buffer (50 mM HEPES pH 7.5, 10% (v/v) Glycerol, 0.5% (w/v) CHAPS, 0.5% (v/v) Triton-X-100, 250 mM NaCl. Prior to use: 1 mM Na3VO4, 1 mM PMSF and protease inhibitor mixture cocktail) and incubated on ice for 30 min under constant agitation on an orbital shaker. Lysates were then centrifuged at 12,000 g for 10 min at 4° C. 80 μl of the supernatant consisting of soluble proteins was transferred to fresh 1.5 ml tubes and stored at −20° C.

### SDS-Polyacrylamide Gel Electrophoresis (SDS-PAGE)

SDS-PAGE was conducted as outlined by Laemmli (Laemmli, 1970) and modified by Studier (Studier, 1973). Samples were denatured in 4× TruPAGE sample buffer at 70° C. for 10 min prior to loading into individual wells. Electrophoresis was performed at a constant 100 V through a 5% SDS polyacrylamide stacking gel and then through a 13% resolving gel at 150 V.

### Immunoblotting

Following separation by electrophoresis, the migrated proteins were transferred electrophoretically to nitrocellulose membranes by a Biorad Wet-Transfer unit. Electrical current (Amps) and transfer times varied depending on the size of the protein of interest. Pre-cut Whatmann paper and nitrocellulose were equilibrated with transfer buffer 10 min prior to use. 3 layers of Whatmann paper were placed on the bottom surface of the transfer unit followed by a single layer of nitrocellulose. The resolving gel was washed briefly in transfer buffer before being placed on the nitrocellulose while avoiding bubbles. 3 more layers of Whatmann paper were placed on top and the unit was closed.

After transfer was completed, membranes were briefly washed with deionised H₂O before incubation with blocking buffer (5% w/v skimmed milk powder in TBST) for 30 minutes on an orbital shaker at RT to inhibit non-specific antibody binding to a membrane. Membranes were then incubated with primary antibody overnight in TBST containing 5% (w/v) skimmed milk powder. The membranes were washed in TBST for 5 minutes under constant agitation. This wash step was repeated 3 times. Membranes were then incubated with a secondary antibody specific to the primary antibody (anti-mouse, anti-rabbit) in TBST containing 5% (w/v) skimmed milk powder at RT for 1 h. The membranes were again washed in TBST 3 times for 5 minutes under constant agitation. Protein bands were then visualised using enhanced chemiluminescence (ECL).

### Enzyme-Linked Immunosorbent Assay (ELISA)

Cell supernatant or serum samples were collected and stored at −80° C. prior to analysis. 96-well NUNC “Maxisorb” plates were coated with 100 μl capture antibody diluted to a working concentration in PBS. Dilutions of capture antibody were lot specific. Plates were covered to avoid evaporation and placed on an orbital shaker overnight at RT. Antibody was aspirated from the plate and each well was washed three times with a wash buffer (0.05% Tween® 20 in PBS). After the final wash, the plate was inverted and blotted against clean paper towels to ensure the removal of excess wash buffer. Plates were blocked by adding 300 μl of block buffer (1% (w/v) BSA in PBS filter sterilised (0.2 m)) for 2 h. Reagent diluent was prepared for the IL-10 plate (0.1% (w/v) BSA in Tris-buffered Saline (20 mM Trizma base, 150 mM NaCl) filter sterilised (0.2 m)) and the TNF-α (1% (w/v) BSA in PBS filter sterilised (0.2 m)). Samples were diluted in reagent diluent and added to each well. Standards were diluted in reagent diluent along a seven-point standard curve using 2-fold serial dilutions with a top standard of 2000 μg/ml. Plates were covered to avoid evaporation and placed on an orbital shaker overnight at 4° C. Plates were washed as above and 100 μl of detection antibody was diluted to a working concentration in reagent diluent. Dilutions of capture antibody were lot specific. Plates were incubated for 2 h at RT and the wash step was repeated. Streptavidin-HRP conjugate was diluted in reagent diluent as per lot specific instructions and 100 μl was added to each well. Plates were incubated in the dark at RT for 20 min and the wash step was repeated. 100 μl TMB (1.25 mM/l) solution was added to each well and the plates were covered and incubated at RT for 20 min. 50 μl 1 N H₂SO₄ stop solution was added to stop the reaction. The optical density (OD) value of each well was measured at 450 nm with a correction at 590 nm. A ELx800™ microplate reader with Gen5 Data Analysis Software was used to carry out the reading. The sample concentrations were determined by interpolating the sample absorbance from the known concentrations of the standard curve. Standards were assayed in duplicate while samples were analysed in triplicate. Data presentation and analysis were carried out using Graphpad Prism 5 software.

### In Vitro Arthritis Model

Enriched inflammatory cell supernatant from DH82 cells was prepared by first stimulating seeded DH82 cells with compound, followed by stimulation with LPS for 3 hours. Media was removed, cells were washed and fresh media was added to cells, in order to avoid any potential crossover of compound into supernatant. After 24 hours, supernatant was removed and added to seeded CNC for 24 hours, in order to simulate an inflammatory joint. After 24 hours, mRNA was harvested from CNC as described above and levels of COL2A1 were assessed.

### In Vivo Techniques

Animals were monitored for adverse clinical signs, matted fur, abnormal behaviour, response to stimulus and weight loss. Any animal with a total cumulative health score exceeding a pre-defined threshold was culled. All mice were culled at the 72 h end-point of the experiment. Data presentation and analysis were carried out using Graphpad Prism 5 software. All experiments were performed in accordance with the regulations and guidelines of the Irish Department of Health and protocols approved by the Research Ethics committee of National University of Ireland Maynooth.

High Fat Diet administration

Age (8-10 weeks) C57BL6 Mice were purchased from Charles River, rehoused and allowed to acclimatise to the new surroundings for one week to avoid stress. Mice were weighed and diet changed to a custom high fat diet (60% fat, enviogen). Mice were weighed weekly for a total of 12 weeks.

### Compound Administration

Age 8 week old mice were initially monitored for a palatability pilot study, where signs of dehydration and weight loss were measured. Compounds deemed palatable and safe were used during the primary study. Mice fed using a high fat diet for 8 weeks were administered compounds of interest in their drinking water. This continued for 4 weeks. Mice were weighed weekly and scored for signs of dehydration. Food and drinking water were weighted weekly to ensure adequate hydration and dosing of compound. Doses varied based on solubility

### Glucose Tolerance Test

On week 12 of high fat diet, food was first removed for 18 hours prior to experimentation. Mouse blood glucose levels were then measured to obtain a baseline glucose level (timepoint 0), using tail bleeds. Mice were then inject intraperitoneally using glucose (1.5 mg/g). Blood glucose was then measured at 15, 30, 60 and 120 min after injection. Food was returned to mice after the final blood sampling.

### Insulin Tolerance Test

Two days after glucose tolerance testing, food was first removed from mice for 5 hours. Mouse blood glucose levels were measured to obtain a baseline glucose level (timepoint 0), using tail bleeds. Mice were then inject intraperitoneally using insulin (0.75 mU/g). Blood glucose was then measured at 15, 30, 60 and 120 min after injection. Food was returned to mice after the final blood sampling.

### Sample Analysis

At the end of week 12 of high fat diet, mice were sacrificed and whole blood, white adipose tissue and liver were extracted. Blood was incubated at RT for 30 minutes, followed by centrifugation at 7500 G for 10 minutes. Serum was collected and stored at −20° C. Serum was analysed using ELISA as described above. Organ samples were homogenised in Trizol and RNA was extracted for real-time PCR analysis as described above.

### Example 1

### Results

All the compounds selected were chosen from an initial list of foods approved for human consumption from the EU and FDA. These lists contain thousands of compounds which offer a great variety of molecular structure. The first step in assessing the capacity for these compounds to have bioactive effects which extends beyond their known function is to consider their molecular structure and potential for biological activity. This builds on in house knowledge of biochemical pathways and in house ability to assess and determine compounds and structures with potential for bioactive effects beyond their known function.

Once selected, the compounds were screened using our cytokine release platform. THP-1 cells were cultured in RPMI (10% FBS, 1% pen/strep) until confluent, at which point cells were counted and seeded into 96 well plates at 1×10⁶/ml. Cells were rested overnight and subjected to varying increasing doses of compounds of interest for 2 hours, with DMSO as the vehicle. Cells were then stimulated with LPS (1 μg/ml) for 24 hours. The next day, cells were pelleted by centrifugation and the supernatant was removed. TNFα and IL-10 were then measured and compared to vehicle alone. Optimal effective doses of compounds to elicit cytokine decrease were determined, allowing us to use these doses for further studies.

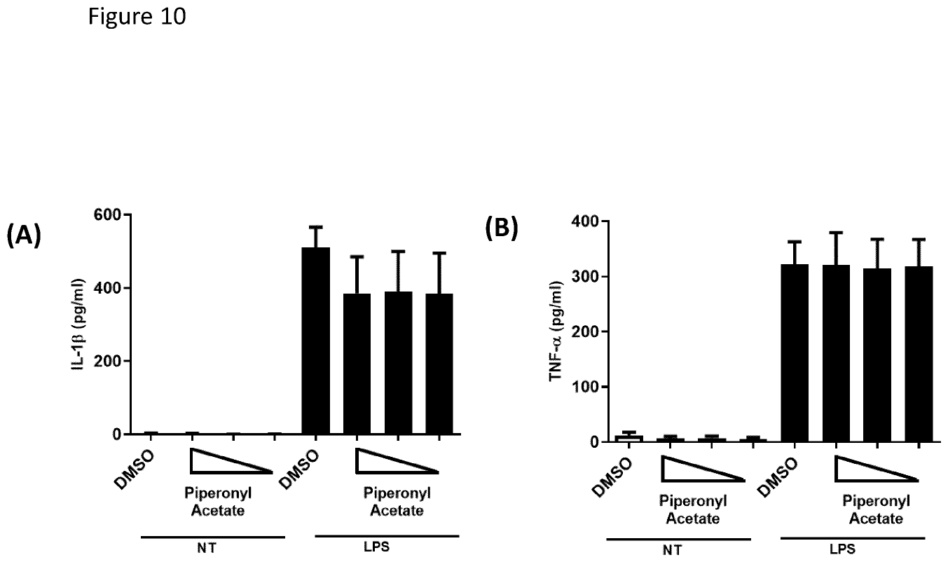

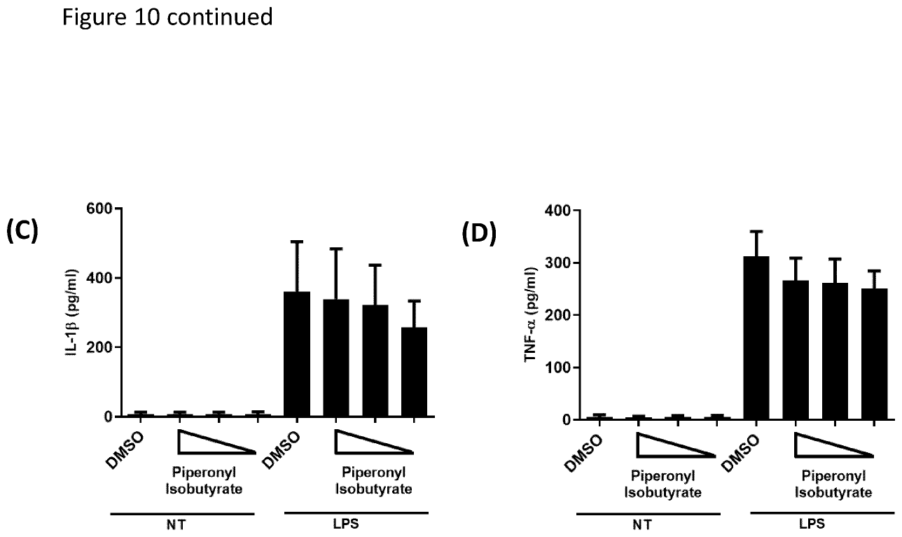

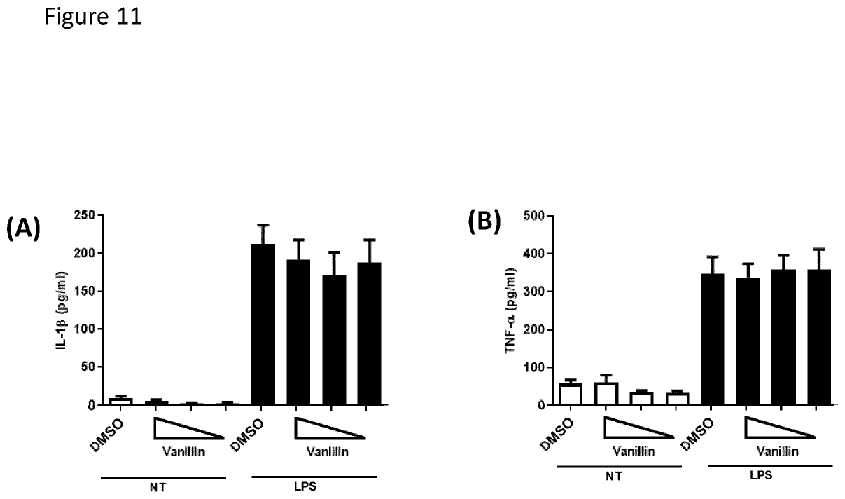

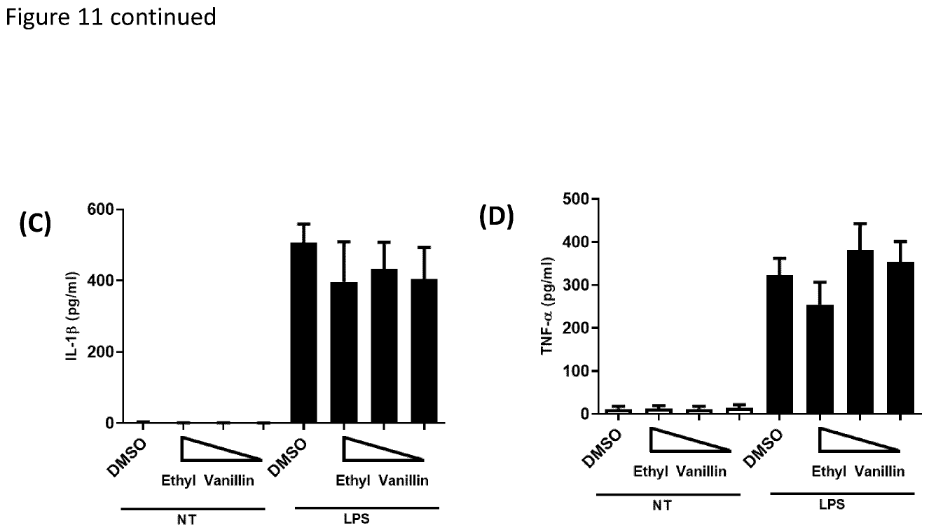

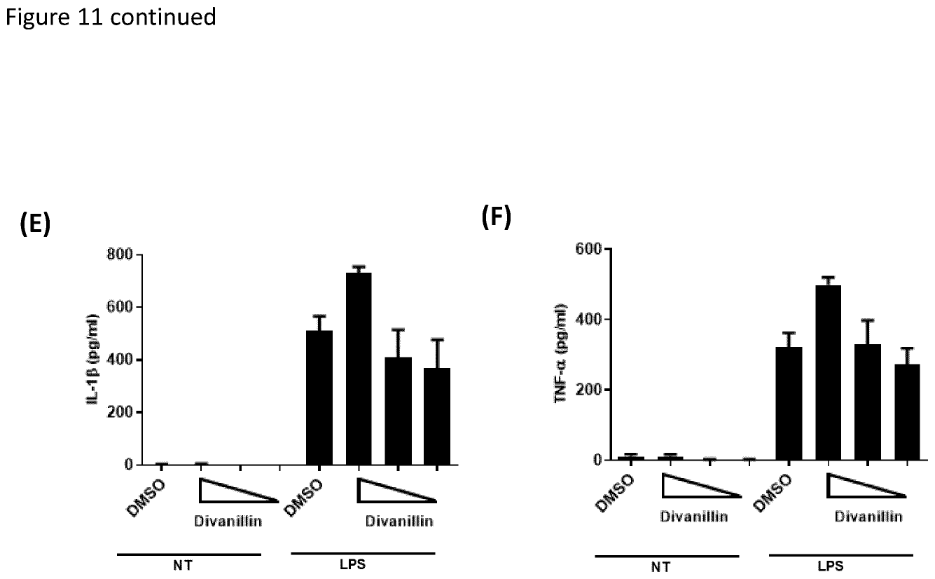

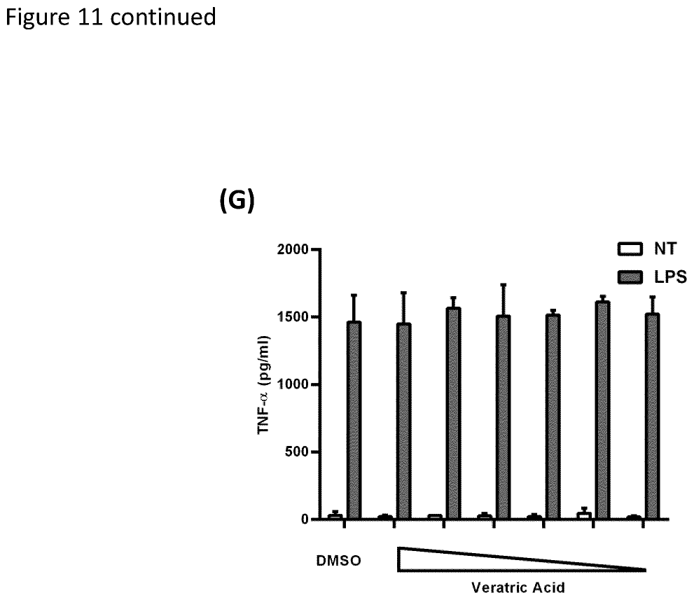

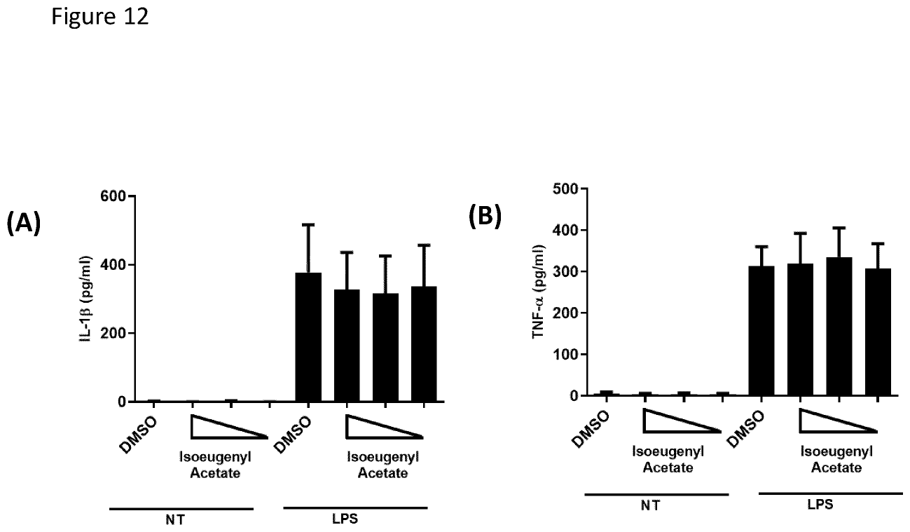

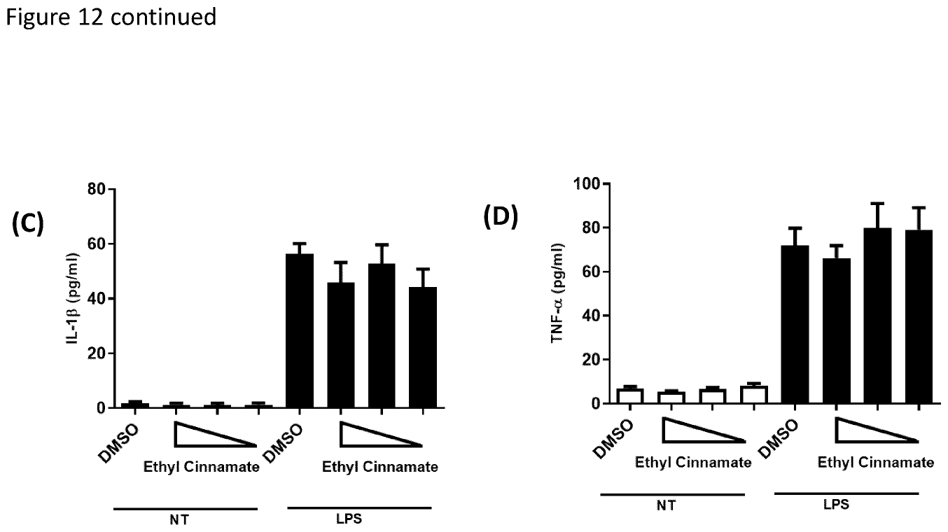

Compounds which successfully hindered the cytokine release of either IL-10 or TNF were then considered for deeper examination. Lead compounds were determined for their suitability in food, effectiveness and lack of prior art in the literature. A number of criteria were used to determine effective compounds, the most important of which is the presence of a statistically significant effect upon addition of compound. A statistical significance is the likelihood that the differences observed between variables is not due to random chance. This was determined by using statistical software. Broadly, the main factors contributing to statistical significance are the sample size and the effect size. A sample size of 3 or more is used in the in vitro portion of testing and the larger the effect size, the more likely significance will be demonstrated. Often, a result may appear by eye to be statistically significant (example FIG. 10A or FIG. 10D), but when analysed using specialist software, there is no apparent significance. This is in part due to the variability between the individual samples and replicates. By choosing an unbiased approach, we were able to confidently move forward with select lead compounds.

The next phase of testing aimed to examine the specificity of the effect of the chosen compounds. Using varying cell types and species allowed us to clarify if the effect we were seeing was THP specific or independent of cell type. Peripheral blood mononuclear cells (PBMC) were used as they allowed us to investigate a primary human cell response. PBMC were treated and seeded as described for THP1. Bone marrow from mice legs were also used in this screening process. This allowed us to test if the compounds were effective independent of species. Bone marrow was extracted from mice, seeded at 2×10⁶/ml in RPMI and rested for two hours prior to stimulation.

The mechanism of action of the compounds of interest were then examined. Initially, messenger RNA was extracted from cells pre-treated with compound and treated with LPS for 3 hours. This gave us an insight into the gene activity of our cells during an immune response. Compounds which showed reduced cytokine release also showed decreased associated gene activity, which indicated that the compounds were exerting their effect prior to gene transcription. Thus, the initial LPS signalling pathway involving TLR4 was examined. It was determined that certain proteins normally phosphorylated during TLR4 signalling lacked this activating signal, indicating that the signalling has been blocked at some point in the pathway.

After performing all in vitro work above, candidate compounds were chosen for further examination in an in vivo model of obesity. C57BL/6 mice were fed using a high fat diet (HFD) for 12 weeks total. On weeks 8-12 of the diet, compounds were introduced to the drinking water of mice, which was consumed ad libitum. On week 12, Glucose tolerance tests and insulin tolerance tests were performed. Mice were sacrificed and serum, liver and adipose tissues samples were taken. Serum was analysed by ELISA, while tissue was prepared for RNA isolation and real time PCR testing. We were able to confirm that a number of compounds which showed effects in vitro were able to affect glucose handling in mice and also lower the TNF gene transcription in the liver.

### Conclusion

We used a bottom up approach to screen compounds which we believed could have an impact on innate immune signalling. We filtered our selections using a variety of in vitro techniques, allowing us to refine our choices prior to in vivo testing in mice. The in vivo experiments verified the in vitro data, giving us confidence in the effectiveness of these compounds to modulate the immune response. The benefit to analysing compounds in this way is that they were already approved for human consumption, which removes safety and regulatory issues normally faced when assessing novel compounds.

### Example 2

### Results

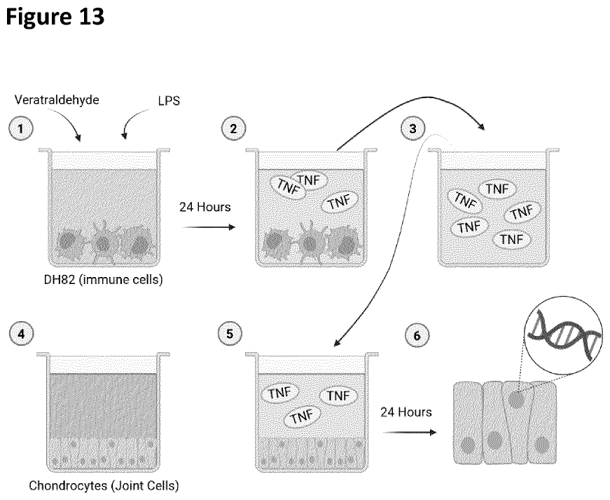

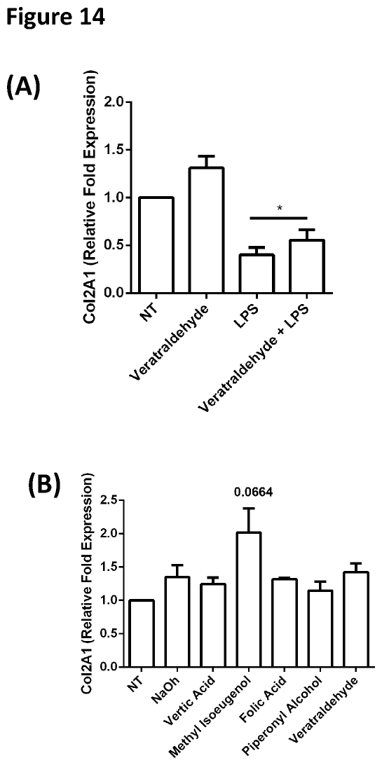

Our previous results demonstrated the potential for our candidate compounds to reduce inflammatory cytokine expression from immune cells. We were further able to demonstrate a phenotypic difference in glucose regulation and TNF production in a murine model of obesity. Next, we aimed to investigate the potential effectiveness of our compounds in other inflammatory disorders, such as arthritis. To accomplish this, we designed an in vitro two cell system which can mimic the inflammatory environment experienced by cartilage cells during disease. We used the gene COL2A1 as a marker for joint health, as COL2A1 is an important gene in the regulation of type II collagen, a key component of cartilage. FIG. 13 demonstrates the method employed to carry out this experiment. FIG. 14(A) shows that when LPS alone is used to induce inflammatory cytokine release from macrophage (immune) cells, there is a large reduction in the levels of the gene COL2A1 in chondrocyte (joint) cells, indicating an arthritic phenotype. However, when macrophage cells are treated with both veratraldehyde and LPS, there is a significant restoration in the levels of COL2A1 when compared to LPS alone. This demonstrates that our compounds could have an effect on improving joint health by reducing the inflammatory conditions in the joint.

FIG. 14(B) shows the effect of an anti-inflammatory agent being used to directly stimulate chondrocyte cells, to see if we are able to induce COL2A1 upregulation, in the absence of any inflammation. The difference here is that, instead of stimulating macrophage cells to produce TNF, as in FIG. 14(A), the anti-inflammatory agents were directly added to the chondrocyte cells to observe the direct effect of compounds on COL2A1 expression.

1. An anti-inflammatory agent for use in preventing or treating an inflammatory disease or condition in a subject, wherein the anti-inflammatory agent is veratraldehyde, or piperonyl alcohol, or ethyl 2-amino benzoate, or isoamyl cinnamate, or diethyl malonate.

2. The anti-inflammatory agent for use according to claim 1 wherein the anti-inflammatory agent comprises veratraldehyde or piperonyl.

3. The anti-inflammatory agent for use according to claim 1 or claim 2 wherein the anti-inflammatory agent is provided as an additive to food for consumption by the subject.

4. The anti-inflammatory agent for use according to any preceding claim, where the anti-inflammatory agent is in a food composition.

5. The anti-inflammatory agent for use according to claim 1 or claim 2, wherein the inflammatory disease or condition is selected from prediabetes, Type 2 diabetes, insulin resistance, gestational diabetes, metabolic disorders, cardiovascular disease (CVD), connective tissue diseases, arthritis and other joint diseases, periodontitis, gout, spondyloarthritis, uveitis, hidradenitis suppurativa, systemic lupus erythematosus (SLE), chronic obstructive pulmonary disease (COPD), psoriasis, rheumatoid arthritis, psoriatic arthritis, inflammatory bowel disease, liver disease, pain, neurological and psychiatric disorders, neurodegenerative disorders (e.g. Alzheimer's, dementia), depression, stress, narcolepsy, COVID 19, viral infections.

6. A pharmaceutical formulation comprising veratraldehyde, or piperonyl alcohol, or ethyl 2-amino benzoate, or isoamyl cinnamate, or diethyl malonate as an anti-inflammatory agent; and
a pharmaceutically acceptable carrier.

7. The pharmaceutical formulation according to claim 8 wherein the formulation is formulated as a powder, liquid, an encapsulated tablet, or an unencapsulated tablet.

8. A method of treating or preventing an inflammatory disorder in a subject comprising oral consumption of an anti-inflammatory agent, wherein the anti-inflammatory agent is veratraldehyde, or piperonyl alcohol, or ethyl 2-amino benzoate, or isoamyl cinnamate, or diethyl malonate.

9. Use of an anti-inflammatory agent in the manufacture of a medicament for use in treating or preventing an inflammatory disorder in a subject,
wherein the anti-inflammatory agent is veratraldehyde, or piperonyl alcohol, or or ethyl 2-amino benzoate, or isoamyl cinnamate, or diethyl malonate.

10. The method according to claim 8, or the use according to claim 9, wherein the anti-inflammatory agent is incorporated into a food product, or provided as a food additive or supplement.

11. A method of making a food product for use in treating or preventing an inflammatory disorder in a subject said method comprising combining an anti-inflammatory agent with a food product, wherein the anti-inflammatory agent comprises veratraldehyde, or piperonyl alcohol, or ethyl 2-amino benzoate, or isoamyl cinnamate, or diethyl malonate.

12. An anti-inflammatory agent for use as a medicament, wherein the anti-inflammatory agent is veratraldehyde, or piperonyl alcohol, or ethyl 2-amino benzoate, or isoamyl cinnamate, or diethyl malonate.

13. The anti-inflammatory agent for use according to claim 12, wherein the anti-inflammatory agent is provided in a form suitable for oral consumption.

14. The anti-inflammatory agent for use according to claim 12 or claim 13, wherein the anti-inflammatory agent is incorporated into a food product or food additive.

15. The anti-inflammatory agent for use according to claims 1-5, the method according to claim 8, the use according to claim 9, and the method for use according to 10 wherein the subject is a human or a non-human animal, optionally wherein the non-human animal is a livestock animal.
